(19) United States
(12) Patent Application Publication
(10) Pub. No.: US 2025/0029705 A1
(43) Pub. Date: Jan. 23, 2025

(54) EVIDENCE-REFERENCED RECOMMENDATION ENGINE TO PROVIDE LIFESTYLE GUIDANCE AND TO DEFINE HEALTH METRICS

(71) Applicant: Joshua Dean Erndt-Marino, Tallahassee, FL (US)

(72) Inventor: Joshua Dean Erndt-Marino, Tallahassee, FL (US)

(21) Appl. No.: 18/907,348

(22) Filed: Oct. 4, 2024

Related U.S. Application Data
(63) Continuation of application No. PCT/US2023/065212, filed on Mar. 31, 2023.
(60) Provisional application No. 63/480,738, filed on Jan. 20, 2023.
(60) Provisional application No. 63/386,722, filed on Dec. 9, 2022.
(60) Provisional application No. 63/327,311, filed on Apr. 4, 2022.

(51) Int. Cl. G16H 20/60; G16H 50/30

(52) U.S. Cl. CPC G16H 20/60; G16H 50/30

(57) ABSTRACT

Systems and methods that can take a plurality of user inputs from various domains, reference this data to external knowledge databases (an evidence-base), process all the variables/information, and provide back to the user: 1) a consensus evaluation of a food's healthfulness, 2) a consensus evaluation of a food's cost effectiveness for human and planetary health, 3) food and diet recommendations consistent with guidelines and user consumption patterns, and 4) predicted effects on a multitude of health outcomes, and their uncertainty, for a multitude of health and lifestyle scenarios do not yet exist. In contrast, for food evaluation, individual systems exist, but are heterogeneous and thus contradictory to one another. Likewise, for health outcomes, systems that provide advice for biomarkers, which are not health outcomes per se, do exist, albeit without providing uncertainty assessment, consistency with existing dietary guidelines, compatibility with belief structures, and/or integration with predictive health models.

### CROSS-REFERENCE TO RELATED APPLICATION

This application is a continuation of, and claims the benefit or priority of, international application PCT/US2023/065212, which has an international filing date of Mar. 31, 2023, which is entitled “EVIDENCE-REFERENCED RECOMMENDATION ENGINE TO PROVIDE LIFESTYLE GUIDANCE AND TO DEFINE HEALTH METRICS,” and which claims the benefit of priority of U.S. provisional patent application No. 63/327,311, titled “Adaptable, Hybrid, Data-Driven and Expert-Knowledge-Informed Diet and/or Food Recommendation System to Optimize Health for Individuals, Populations, and Planet,” filed on Apr. 4, 2022, U.S. provisional patent application No. 63/386,722, titled “Evidence-Based Recommendation Engine,” filed Dec. 9, 2022, and U.S. provisional patent application No. 63/480,738, titled “Evidence-Based Recommendation Engine,” filed Jan. 20, 2023. The contents of all above-referenced applications, in entirety, are incorporated herein by this reference.

### TECHNICAL FIELD

The present disclosure relates an evidence-based recommendation engine to rate foods consistent with multiple food ratings systems, dietary guidelines, clinical dietary interventions, health outcomes, and user consumption patterns and to evaluate and communicate individual- and/or population-level health outcome risk scenarios quantitatively, graphically, schematically, and qualitatively.

### BACKGROUND

Individuals, subpopulations, and populations make lifestyle choices, including diet and food based, nearly every minute of each day throughout their lifetime, often motivated by their perceived or predicted effects on a future health state and/or based on their alignment with published, external guidelines provided by trusted sources. Yet, major gaps exist in formalizing this process to augment various desirable features such as decision efficiency, prediction precision or accuracy, lifestyle intervention prioritization, compatibility with existing belief structures (e.g., alignment with a trusted dietary guideline or a lifestyle preference), and adaptive reference to external databases/knowledge/results from a large body of scientific studies.

### SUMMARY

This summary is provided to briefly introduce concepts that are further described in the following detailed descriptions. This summary is not intended to identify key features or essential features of the claimed subject matter, nor is it to be construed as limiting the scope of the claimed subject matter.

In at least one embodiment, a system for providing a food and dietary recommendation and corresponding predicted effects profile includes: at least one processor; a communication interface communicatively coupled to the at least one processor; and a memory device storing executable code. When executed, the code causes the processor to: receive, from a user device, user-specific personal data comprising user-specific biological metrics; receive, from the user device, user selection inputs comprising user-specific dietary and food consumption data; receive, from the user device, user selection inputs comprising user-specific health outcome and prediction model preference data; automatically generate and store, in a system-internal database, a user-specific health and consumption profile based at least on the received user-specific personal data and user selection inputs; access at least one external database and retrieve therefrom evidence-based data associated with at least portions of the user-specific health and consumption profile; generate a food recommendation data object based at least in part on the user-specific biological metrics and retrieved evidence-based data; generate a dietary recommendation data object based at least in part on the user-specific biological metrics and retrieved evidence-based data; generate, by predictive modeling, a predicted effect data object based at least in part on the dietary recommendation data object and the user-specific biological metrics; and transmit for display, at least in part, on the user device: the food recommendation data object; the dietary recommendation data object; and the predicted effect data object.

In at least one embodiment, a method is provided for a computing system to provide a food and dietary recommendation and corresponding predicted effects profile. The computing system includes one or more processor, and at least one memory device storing computer-readable instructions, the one or more processor configured to execute the computer-readable instructions thereby causing the processor to: receive, from a user device, user-specific personal data comprising user-specific biological metrics; receive, from the user device, user selection inputs comprising user-specific dietary and food consumption data; receive, from the user device, user selection inputs comprising user-specific health outcome and prediction model preference data; automatically generate and store, in a system-internal database, a user-specific health and consumption profile based at least on the received user-specific personal data and user selection inputs; access at least one external database and retrieve therefrom evidence-based data associated with at least portions of the user-specific health and consumption profile; generate a food recommendation data object based at least in part on the user-specific biological metrics and retrieved evidence-based data; generate a dietary recommendation data object based at least in part on the user-specific biological metrics and retrieved evidence-based data; generate, by predictive modeling, a predicted effect data object based at least in part on the dietary recommendation data object and the user-specific biological metrics; and transmit for display, at least in part, on the user device: the food recommendation data object; the dietary recommendation data object; and the predicted effect data object.

The below examples are provided with respect to both the above system and method embodiments. It is to be understood that the received user inputs may previously or concurrently be requested, solicited, prompted, or otherwise permitted by way of questions, queries, and/or entry opportunities enabled, for example, by way of a graphical user interface and/or other display and output elements sent to or otherwise caused at user devices and outputs thereof such as displays. For example, a graphical user interface may be caused at a user device by which the inputs may be entered and thereby ultimately received.

In at least one example, the predictive modeling is conducted by back propagating model parameters through a food composition database and generating both absolute and relative health-effect predictions and food scorings.

In at least one example, said back propagation is performed through posterior predictive distributions from selected models or from summary statistics of the parameters.

In any above embodiment and example, predictive health model(s) may be used to quantitatively compare and prioritize multiple competing lifestyle interventions in terms of their capacity to affect a future health state, to be further evaluated with randomized controlled clinical trials.

In any above embodiment and example, multiple food scoring systems may be aggregated to generate the food recommendation data object.

In any above embodiment and example, wherein a respective uncertainty may be transmitted with each of the food recommendation data object, the dietary recommendation data object, and the predicted effect data object.

In any above embodiment and example, to generate the food recommendation data object, the dietary recommendation data object, and the predicted effect data object, the processor may use data-driven scoring systems, and expert-consensus based scoring systems.

In any above embodiment and example, to generate the food recommendation data object, the dietary recommendation data object, and the predicted effect data object, the processor may utilize user-specific relative beliefs.

The above summary is to be understood as cumulative and inclusive. The above described embodiments, features, and examples are combined in various combinations in whole or in part in one or more other embodiments.

### BRIEF DESCRIPTION OF THE DRAWINGS

The previous summary and the following detailed descriptions are to be read in view of the drawings, which illustrate some, but not all, embodiments and features as briefly described below. The summary and detailed descriptions, however, are not limited to only those embodiments and features explicitly illustrated.

FIG. 1, representing a core workflow of a system or framework of the invention, depicting where outputs are generated from user inputs, and the overall architecture of the modules, according to at least one embodiment, is intended to be viewed in combination with FIGS. 2, 3, and 8-24 as indicated.

FIG. 2, representing a second portion of the system or framework is intended to be viewed in combination with FIG. 1 as indicated, shows an example user interface with a subset of variables/information that are learned from the user and passed into the respective user databases for storage and subsequent processing.

FIG. 3 represents, in some embodiments, detail on external databases (evidence-base) and where they fit within the engine and within each module, and is intended to be viewed in combination with FIGS. 1, 4-6 as indicated, in which externally derived databases (available from existing literature or public resources) are in rectangles throughout.

FIG. 4 represents or illustrates further steps or examples of implementation specifically focusing on the food and diet processing unit, at least each in part, of systems and methods described herein.

FIG. 5 represents or illustrates further steps or examples of implementation specifically focusing on the risk factor scenario generator, at least each in part, of systems and methods described herein.

FIG. 6 represents or illustrates further steps or examples of implementations specifically focusing on the predictive health model(s) unit, at least each in part, of systems and methods described herein.

FIG. 7 represents an example scoring sheet containing the logic underpinning a clinical dietary guideline or dietary pattern or dietary quality index known as the Dietary Approaches to Stop Hypertension (DASH) diet.

FIG. 8 represents an example of Output 1, an evaluation of the food supply rated based on dietary index logic to measure consistency with the guideline (shown by food category), and is intended to be viewed in combination with FIGS. 1, 4 as indicated.

FIG. 9 represents an example of Output 2, a display of food identification cards which recommend foods to be encouraged (e.g., that are complemented to the user's diet quality adherence subcomponents) or avoided (e.g., anti-complemented), showing the whole food supply, and is intended to be viewed in combination with FIGS. 1, 4 as indicated.

FIG. 10 represents an example of Output 2, a display of food identification cards that are shown by a food category which recommend foods to be encouraged or avoided, and is intended to be viewed in combination with FIGS. 1, 4 as indicated.

FIG. 11 represents an example of Output 3, a spider plot (e.g., radar chart, circular bar graph) depicting adherence to, or consistency with, an expert-derived dietary quality index or guideline, including its components, for the optimal risk factor scenario, and is intended to be viewed in combination with FIGS. 1, 4 as indicated.

FIG. 12 represents an example of Output 3, a spider plot depicting a food recommendation (e.g., white rice, steamed) and how this food would improve adherence to, or consistency with, an expert-derived dietary quality index or guideline, including its components, and is intended to be viewed in combination with FIGS. 1, 4 as indicated.

FIG. 13 represents an example of Output 4, graphical (e.g., speedometer charts, radar charts, circular bar graphs, density charts, histograms) or tabular displays of health or risk status under various lifestyle scenarios for individuals and/or populations, and is intended to be viewed in combination with FIGS. 1, 6 as indicated.

FIG. 14 represents an example of Output 4, graphical (e.g., speedometer charts, radar charts, circular bar graphs, density charts, histograms) or tabular displays of health or risk status under various lifestyle scenarios for individuals and/or populations, and is intended to be viewed in combination with FIGS. 1, 6 as indicated.

FIG. 15 represents an example of Output 4, graphical (e.g., speedometer charts, radar charts, circular bar graphs, density charts, histograms) or tabular displays of health or risk status under various lifestyle scenarios for individuals and/or populations, and is intended to be viewed in combination with FIGS. 1, 6 as indicated.

FIG. 16 represents an example of Output 4, graphical (e.g., speedometer charts, radar charts, circular bar graphs, density charts, histograms) or tabular displays of health or risk status under various lifestyle scenarios for individuals and/or populations, and is intended to be viewed in combination with FIGS. 1, 6 as indicated.

FIG. 17 represents the process of constructing, evaluating, and selecting predictive health models for a health outcome and population of potential interest to a user—biological age in the general United States population, and is intended to be viewed in combination with FIGS. 1,6 as indicated, and also showcases how models constructed using a subset of nutrition facts panels can be simultaneously benchmarked against gold-standard models from nutrient profiling systems or diet quality indices.

FIG. 18 represents the process of constructing, evaluating, and selecting predictive health models for a health outcome and population of potential interest to a user—global cognition in the general United States population of individuals aged 60+, and is intended to be viewed in combination with FIGS. 1,6 as indicated, and further showcases how models constructed using a subset of nutrition facts panels can be benchmarked against gold-standard expert-derived models, and shows how important social determinants of health (e.g., education) may be uncovered and quantified.

FIG. 19 represents an example of Output 5, an evaluation of food healthfulness based on a data-driven nutrient profiling system tailored to specific health conditions (e.g., cognitive health) and is intended to be viewed in combination with FIGS. 1, 6, and 18 as indicated, and explicitly shows the innovative process of parameter backpropagation to use predictive models to rate foods in terms of both absolute and relative health effects.

FIG. 20 represents an example of Output 5, an evaluation of food healthfulness based on a data-driven nutrient profiling system tailored to specific health conditions (e.g., cognitive health), and is intended to be viewed in combination with FIGS. 1, 6, 18, and 19 as indicated, and shows the result when the process in FIG. 19 is extended to a food composition database containing a list of food items in the food supply.

FIG. 21 represents further steps of implementation, at least each in part, of systems and methods described herein, playing particular emphasis on integrating both expert-derived food ratings with data-driven food ratings tailored to specific conditions, further overlaying this consensus healthfulness evaluation with planetary health metrics and cost; the end result is a meta-nutritional cost-effectiveness index for individual foods; and the meta-nutritional profiling processing unit is meant to be viewed in combination with FIGS. 1, 4, and 6 as indicated.

FIG. 22 represents the process of harmonizing multiple heterogeneous food rating systems derived from either expert-NPS, dietary-guideline based NPS, and/or data-driven, health outcome specific NPS; the end result is the healthfulness Recommendation Index, representing highly rated foods with high confidence consistent with the users' preferences in terms of health outcomes and relative weights between the two subsystems (expert derived vs. the data-driven NPS).

FIG. 23 represents one of the outcomes from FIGS. 21 and 22, viewing the degree of heterogeneity across multiple heterogeneous food rating systems, including how a single food can be viewed in combination with and reference to the distribution of food ratings for the food supply for multiple systems.

FIG. 24 represents an example of Output 6, providing a consensus evaluation of food items in terms of human and planetary healthfulness; an entire food supply is shown graphically, with one food overlaid to exemplify the ability to recommend individual foods.

FIG. 25 is a flow chart representing a method, according to at least one embodiment, for a computing system to provide a food and dietary recommendation and corresponding predicted effects profile.

FIG. 26 diagrammatically represents at least one system for implementation of methods according to these descriptions.

FIG. 27 diagrammatically represents at least one device for implementation of methods according to these descriptions.

### DETAILED DESCRIPTIONS

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although steps may be expressly described or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

Like reference numbers used throughout the drawings depict like or similar elements. Unless described or implied as exclusive alternatives, features throughout the drawings and descriptions should be taken as cumulative, such that features expressly associated with some particular embodiments can be combined with other embodiments.

Unless defined otherwise, technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a,” “an,” and “the” refer to “one or more” when used in the subject specification, including the claims. Unless indicated to the contrary, the numerical parameters set forth in the instant specification and attached claims are approximations that can vary depending upon the desired properties sought to be obtained within the scope of these descriptions.

The below descriptions and drawings therewith detail systems and methods, which can take a plurality of user inputs from various domains, reference this data to external knowledge databases (an evidence-base), process all the variables/information, and provide back to the user: 1) food and diet recommendations consistent with multiple food rating systems, dietary and clinical guidelines, health outcomes, and user consumption patterns, and 2) predicted effects on a multitude of health outcomes, and their uncertainty, for a multitude of health and lifestyle scenarios does not yet exist. In contrast, systems that provide advice for biomarkers, which are not health outcomes per se, do exist, albeit without providing uncertainty assessment, consistency with existing dietary guidelines, integration of multiple conflicting food ratings, compatibility with belief structures, and/or integration with predictive health models.

From a consumer's perspective, how do we know which lifestyles are healthiest? Within food, how can we parse all of the conflicting evidence and food rating systems? How much of our intuition is supported by evidence? The present invention helps answer these questions through a simple 3-step process. First, they tell the system about themselves by filling out a survey and uploading existing data they have from a variety of other sources into a computer. Second, they can select a health outcome of interest (e.g., heart health, hypertension, or depression) or many outcomes of interest. Part of the invention uniquely analyzes these data from the first two steps and provides an assessment of the current health of the user as one scenario, including outcomes they are most at risk for. In addition to this ‘current health’ scenario and prioritization, the invention also displays the expected effect of changing to other lifestyle scenarios on the future health of the individual or a population (a group of individuals) using prediction models. The recommendations are shaped by the user's beliefs and supported by an evidence-base, which can include data from the user(s) along with existing scientific studies, data from clinical trials, data reflecting published dietary guidelines from trusted health organizations, data from studies assessing the healthfulness of foods, and data about the quality of the health prediction models. In the third and final step, the recommendations are delivered to the user in a variety of graphical, tabular, quantitative, and qualitative formats to help with decision making and understanding which lifestyles are the healthiest.

The below paragraphs having first-line reference numbers describe referenced elements in the drawings.

102—From dashboard, or webpage, or mobile device: An interactive user interface that is utilized to extract specific information from individuals.

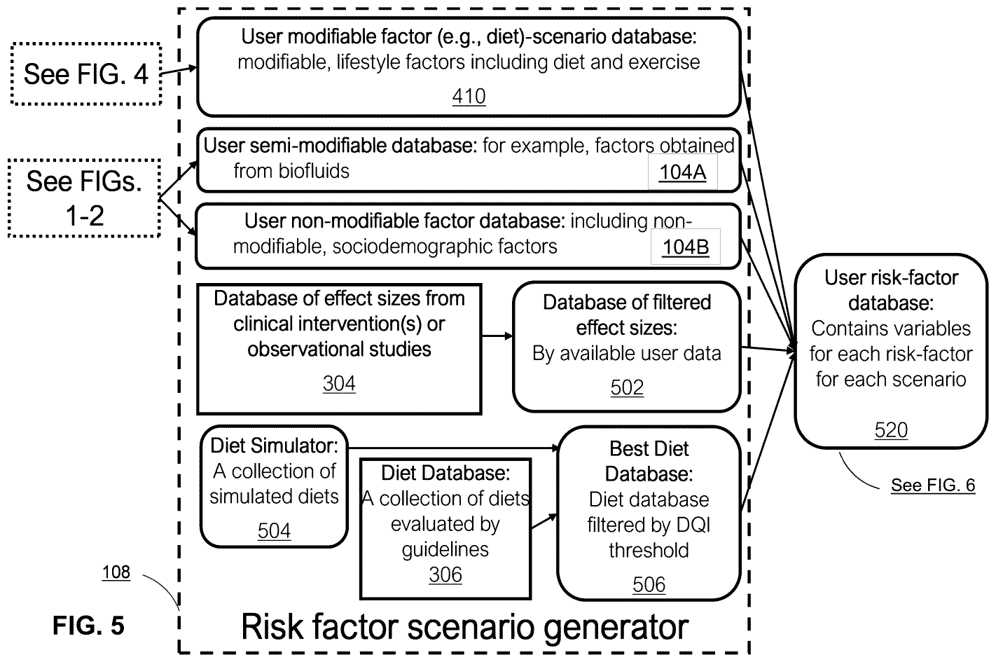

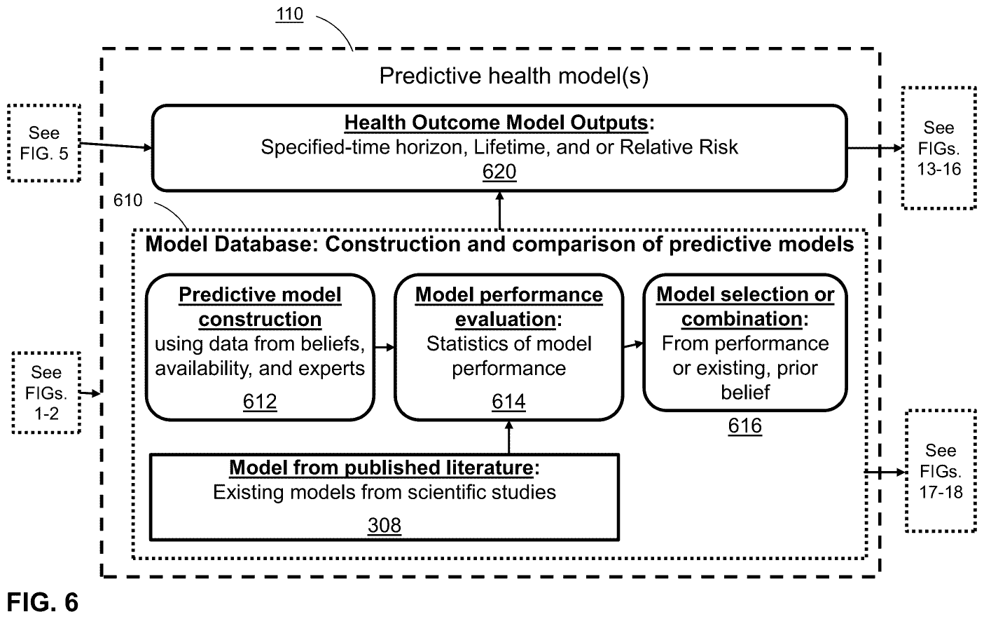

104—User database(s): A collection of information extracted from 102 which can include the following: Non-modifiable factor database, Semi-modifiable factor database, Modifiable factor database, and a Belief Structure database. Variables within each database can include, but are not limited to:
Non-modifiable factors: (see 104B, FIG. 5) Include a collection of variables that can not be changed such as age, birth sex, race/ethnicity, and genetics (not including epigenetics),
Semi-modifiable factors: (see 104A, FIG. 5) Include a collection of variables that can be partially changed such as
   Some environmental: education level, income level, location of residence,
   Physiological readouts from various metrics and tests:
      Non-invasively acquired from existing devices such as blood pressure (systolic and diastolic), heart-rate (and its variability), pulse pressure, blood oxygenation,
      Chemical factors (e.g., triglycerides, HDL, LDL and total Cholesterol, etc. . . . ) measured in biofluids such as blood, plasma, saliva, urine, stool, and interstitial tissue fluid,
      Tissue status obtained from non-invasive imaging modalities such as X-ray, computed tomography, positron emission tomography, magnetic resonance imaging, ultrasound, near infrared spectroscopy, tissue status obtained from molecular and structural analysis of tissue biopsies such as fluorescence microscopy, ELISA, MAGPIX, and immunohistochemistry,
Modifiable factors: Include a collection of lifestyle factors including diet, exercise (physical activity) and social environment.
   Diet: Measured by self-reported 24 h recalls or food frequency questionnaires and can include the quantities and timing of the intake of food- and nutrient-level variables, or an aggregated diet quality index (DQI) score.
      Food variables include but are not limited to fruits, vegetables, processed meats, beans/legumes, nuts and seeds, fruit juices, sugar sweetened beverages, potatoes, etc. . . .
      Nutrient variables include but are not limited to sodium, potassium, magnesium, calcium, iron, selenium, copper, carbohydrates (and constituent sugars), added sugars, protein (and constituent amino acids), fats (including total, trans, poly- and mono-unsaturated, saturated, and constituent fatty acids), alcohol (including constituent chemical varieties),
      Diet quality measured by diet quality index (DQI) scores or adherence to dietary guidelines includes but is not limited to DASH, MIND, HEI, aHEI, MedD, low-sodium DASH, low-carbohydrate DASH, Ketogenic,
   Physical activity measured by recorded activities and their duration (e.g., yoga for 45 minutes, vigorous strength training for 1 h, etc.) or their converted MET hours, and
   Social environment such as time and nature spent reading articles, doing puzzles, socializing with friends, etc.
Belief Structures: Preference of specific predictive models for a given health outcome (e.g., the user selects the Framingham risk score for cardiovascular disease and heart health) and/or the evaluation criteria and statistics by which one or many predictive models would be selected (shown in FIG. 6) from a database of predictive models for one or many health outcomes (shown in 610), health outcome preference (e.g., value heart health at 20% and brain health at 80%), health information delivery preference (e.g., Specified-time horizon risk/health event or value such as 10 y from current time, Lifetime Risk such as the probability of an event or value happening for the remainder of user's life, or Relative Risk such as percentile rank vs. a reference population), dietary preferences (e.g., avoid fish, adhere to Mediterranean or Vegetarian diet pattern, etc. . . . ), a prior expectation or distribution representing the relative or absolute effect size of any modifiable factor (e.g., physical activity is not important but dietary patterns such as DASH are the healthiest, DASH can have an effect of reducing blood pressure by an average of 10 mmHg with variance of 2 mmHg, etc.)

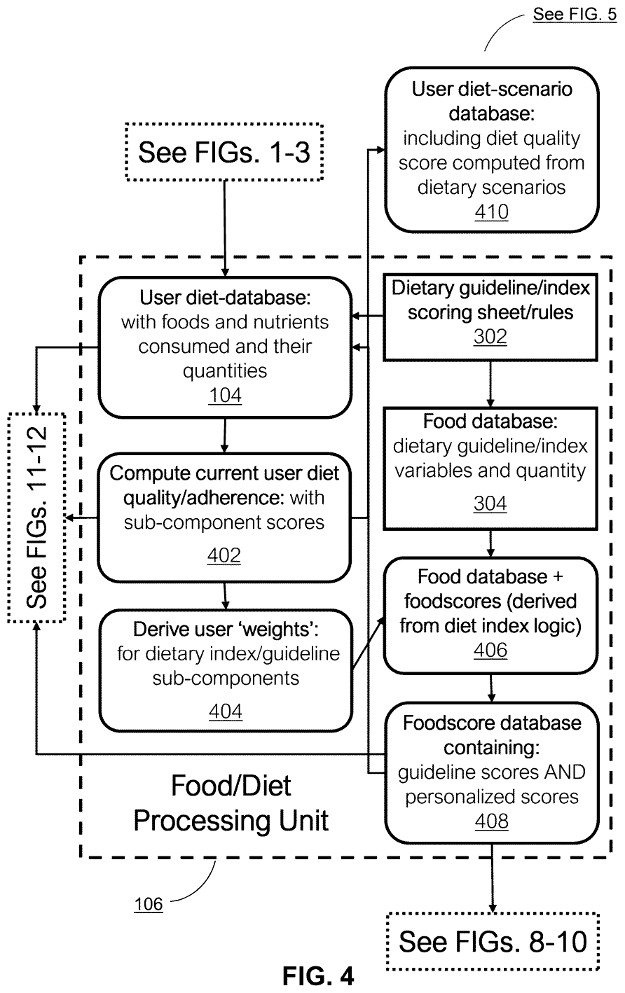

106—Food and diet processing unit: A collation of databases and computational processes that take user data from stored database(s), reference this data to an existing evidence-base, and output a number of graphics and processed data back to the user or to subsequent processing steps. This is explained in detail in FIG. 4.

108—Risk factor scenario generator: Any combination of variables and their levels present in the user database(s) can be matched and referenced to effect sizes from clinical trials, which are stored in a database 304. This is shown in detail in FIG. 5.

110—Predictive health model(s): A process for creating, storing, fitting, analyzing/evaluating, selecting, and combining predictive model(s) for a health outcome or multitude of health outcomes. Any part of the model, including its parameters and their uncertainty, determined by either simulating from a summary distribution or with samples drawn directly from their joint posterior predictive distribution, can be stored and utilized to generate predictions, in some forms using inputs directly from the risk factor scenario database. The prediction outputs may be stored, displayed, and communicated in a variety of formats, as shown in detail in FIG. 6.

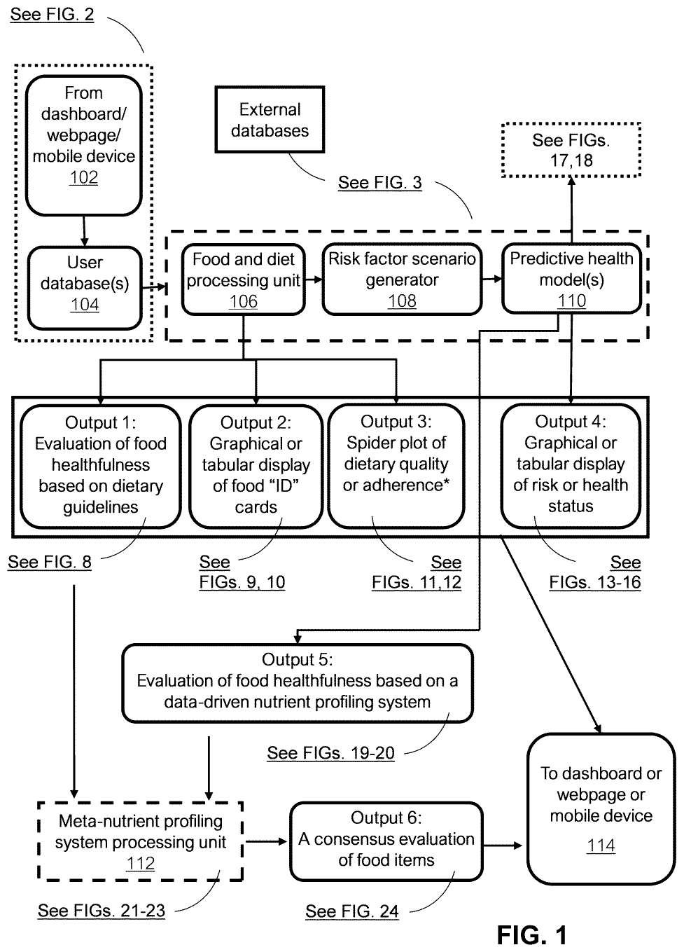

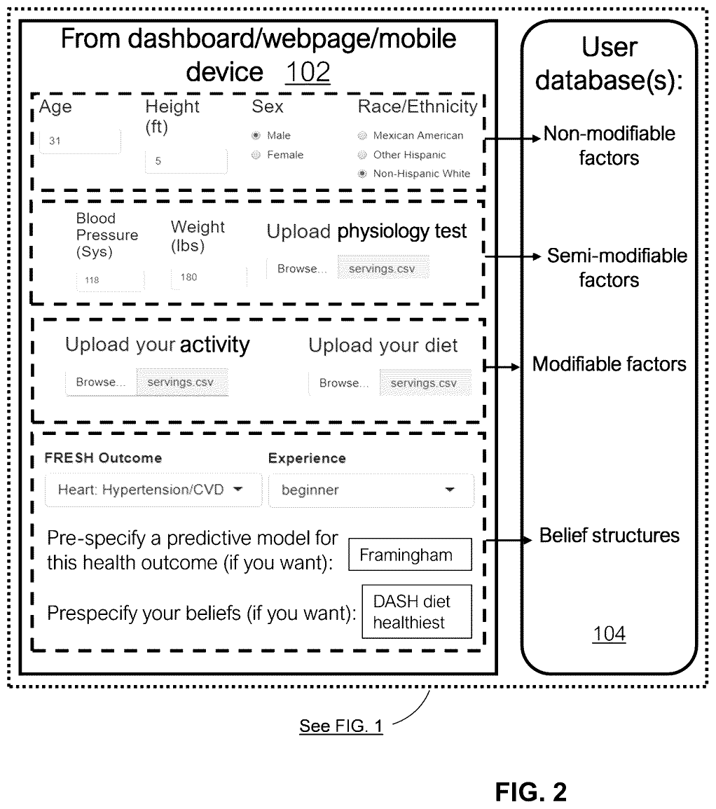

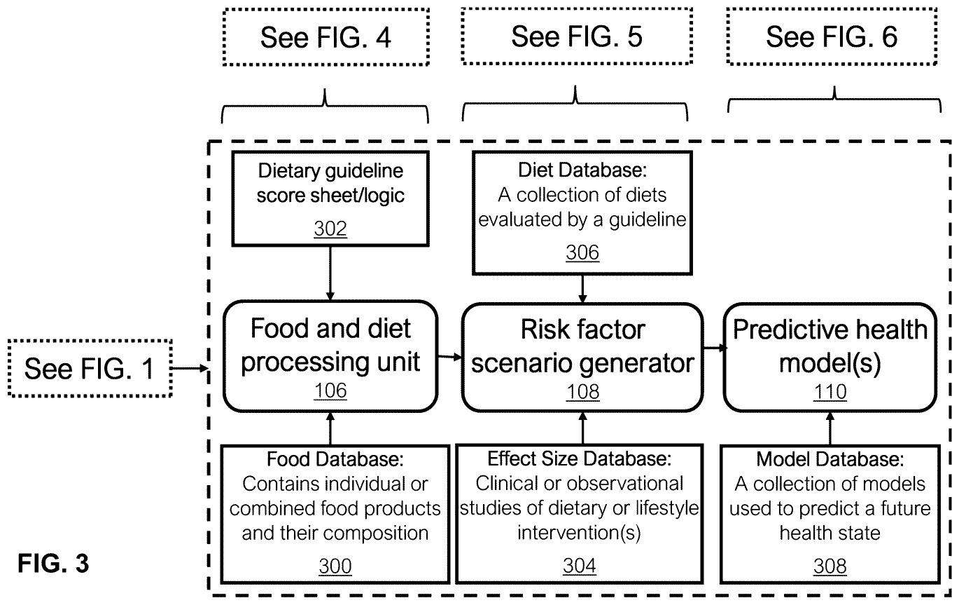

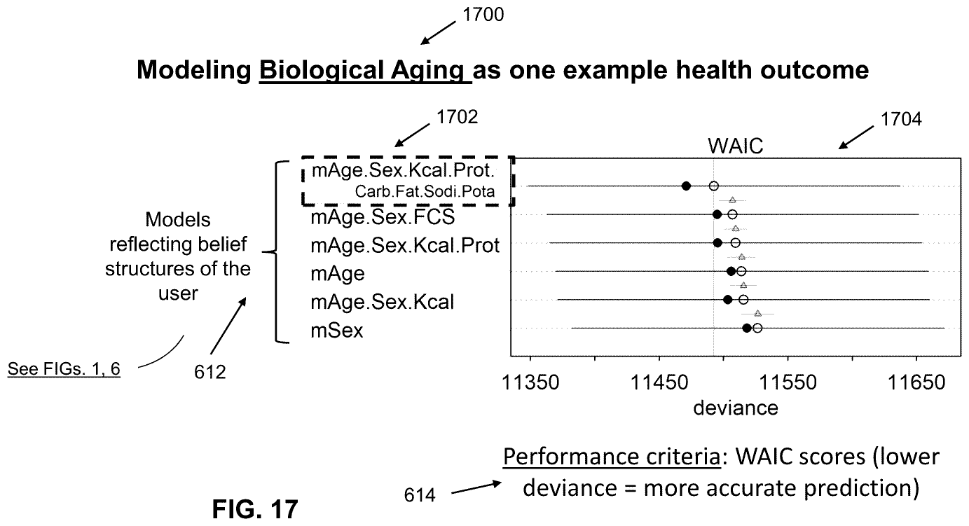

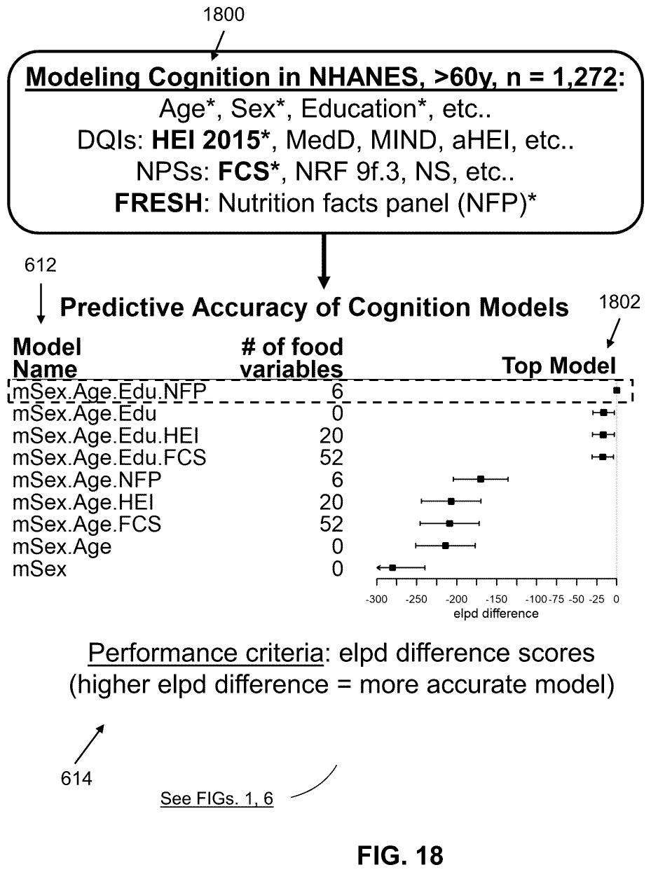

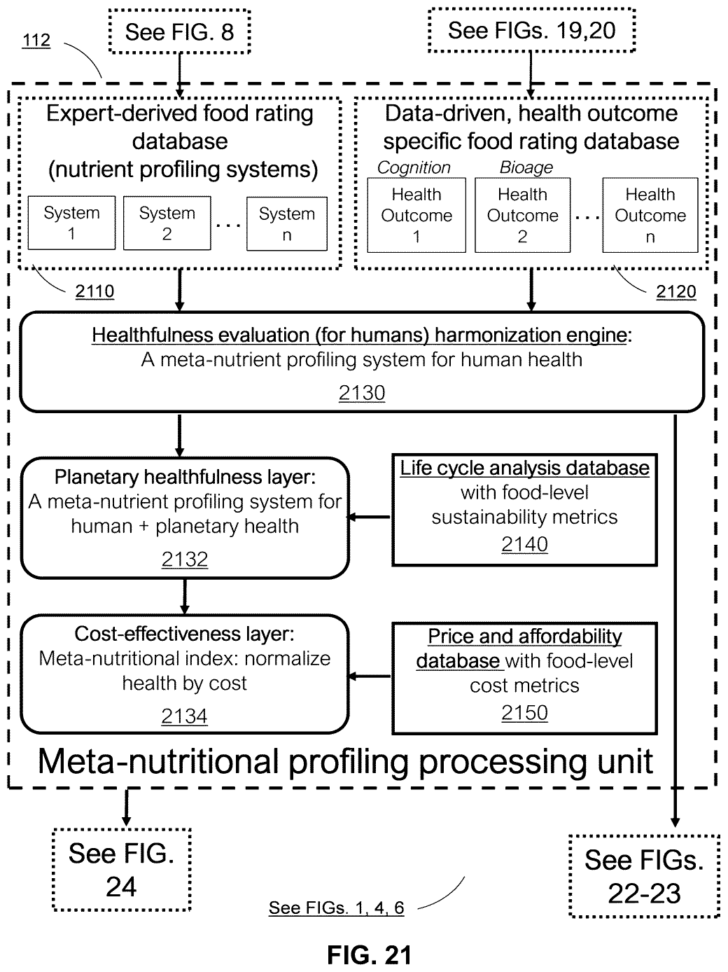

112—Meta-nutritional profiling system: With general reference to FIGS. 1-6, which together diagrammatically represent inventive methods and systems described herein, food recommendations are made with considerations encompassing multiple scoring systems, each having unique strengths and weaknesses, and these are integrated into a meta-nutritional profiling system, which has both expert-derived consensus food recommendation systems (i.e., a nutrient profiling system—NPS) 2110 that may be paired with de novo scoring systems with health outcome specific data-driven recommendations 2120, which are derived from predictive health model(s) in FIG. 6 (element 610) and its derivatives FIGS. 17-18. So rather than, for example, a team of scientists and others deciding what their collective opinion is for what factors to include and how to weigh, score, and combine them, the inventive methods and systems described herein utilize data from real people, their dietary intakes, their non-modifiable and semi-modifiable factors, their health preferences, and their measured outcomes. If, for example, an individual prefers to be advised toward foods that would reduce the risk of depression, with less emphasis or concern about cardiovascular disease, that kind of preference can be taken into account. More detail is provided in FIG. 21 and the figures cited within.

300—Food Database: A collection of individual food products and their composition and other taxonomical information such as brand name, store location and availability. In terms of composition, this can include, but is not limited to, quantities of nutrients, food-groups, ingredients, and other chemicals. As non-limiting examples, nutrients may include potassium, sodium, magnesium, cholesterol, carbohydrate (and carbohydrate composition), protein (and amino acid composition, and fat (and fatty acid composition).

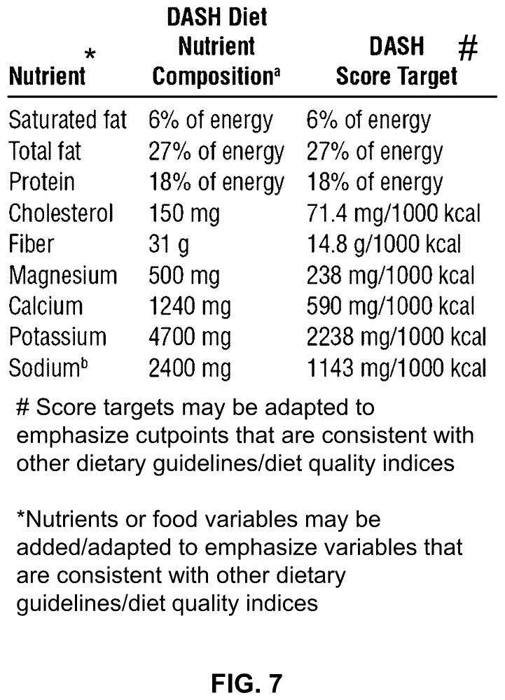

302—Dietary guideline/index scoring sheet/rules: including nutrient- or food-targets to meet for an optimal diet. FIG. 7 shows an example dietary guideline/dietary quality index (DQI) scoring sheet that contains rules for the Dietary Approaches to Stop Hypertension (DASH) diet.

304—Effect Size Database: Clinical, randomized controlled trials of dietary intervention(s): Can also include effect sizes from observational studies and be expanded to any lifestyle intervention (physical activity+diet, physical activity only, etc. . . . ) and include subcomponents of physical activity or diet (e.g., yoga, strength training, aerobic or anaerobic exercise, high fruit intake, high vegetable intake, etc. . . . ) or any diet quality index (e.g., DASH, MedD, HEI, etc. . . . ). These effect sizes from randomized RCTs or observational studies may be combined (e.g., pooled meta-analytically or otherwise with formal statistical rules) or be kept as effect sizes of interventions from individual trials and observational studies.

306—Diet Database: A collection of individuals' diets evaluated by the dietary guideline/DQI. Can be a clinically used index to represent a dietary pattern designed for a clinical health condition, including but not limited to DASH, low-sodium DASH, low-carbohydrate DASH, high-(mono- and poly-) unsaturated fatty acid DASH, Mediterranean-DASH diet for Neurodegenerative Delay (MIND) diet.

308—Model Database from published literature sources: A collection of models used to predict a future health state, outcome, or status. Examples for cardiovascular conditions and health can be the Framingham score, QRISK, SCORE2, and Pooled Cohort Equations. There are also lifetime risk models that can be pulled from scientific literature.

402—Compute current user diet quality/adherence: including sub-component scores, a database of user diet-data that has been processed and evaluated by the dietary guideline scoring logic which has sub-components that are first scored and then averaged or summed, depending on the rules of the index or guideline.

404—Derive user ‘weights’ for dietary index/guideline sub-components: The inverse of the component adherence is used to generated weights that represent the relative deficiency or gap in a given dietary component in the user's reported diet. These weight vectors are utilized to identify foods that complement or anti-complement the user's diet which contributes to providing personalized outputs and food recommendations tailored to the user's consumption patterns. The weights can be further refined by user preferences for single or groups of components to prioritize.

406—Food database+foodscores (derived from diet index logic): User weight vectors are passed into the food database with pre-scored foods and utilized to rescore these same foods using personalized weights determined from 404.

408—Foodscore database containing guideline scores and personalized diet quality index scores, along with food names and the compositional variables and their quantities needed to evaluate the food according to the guidelines.

410—User diet-scenario database: includes a diet quality score as computed from dietary scenarios.

502—Database of filtered effect sizes: By available user data (at maximum) or further by user preference/belief structure learned with graphics.

504—Diet Simulator: A collection of simulated diets evaluated by the dietary guideline/DQI or by the data-driven predictive health models.

506—Best Diet Database: Diet database filtered by DQI threshold above a user-preference feature. Always include the best diet (>90% optimal).

520—User risk-factor database: Contains variables for each risk-factor for each scenario; including predicted changes to the user's data based on effect-sizes from existing randomized, controlled clinical trials. Notes which factors are considered modifiable, nonmodifiable, and semi-modifiable.

610—Model Database: Construction and comparison of predictive models, comprising subcomponents 612-616, and reference to an external, publicly available database 308. These models are directly utilized to generate data-driven nutrient profiling systems tailored to health outcomes and are further processed according to the user to provide health outputs in 620. The model database also includes statistics of each model to evaluate performance, including but not limited to elements described in 614. As represented in FIGS. 1, 3 and 6, methods and systems described herein, in some embodiments, implement a machine learning approach which contains a database of multiple models 610, which comprises constructing statistical models that are (probabilistically) determinative or predictive of risk factor impacts on certain health outcomes/physiological metrics 1700, considering many factors.

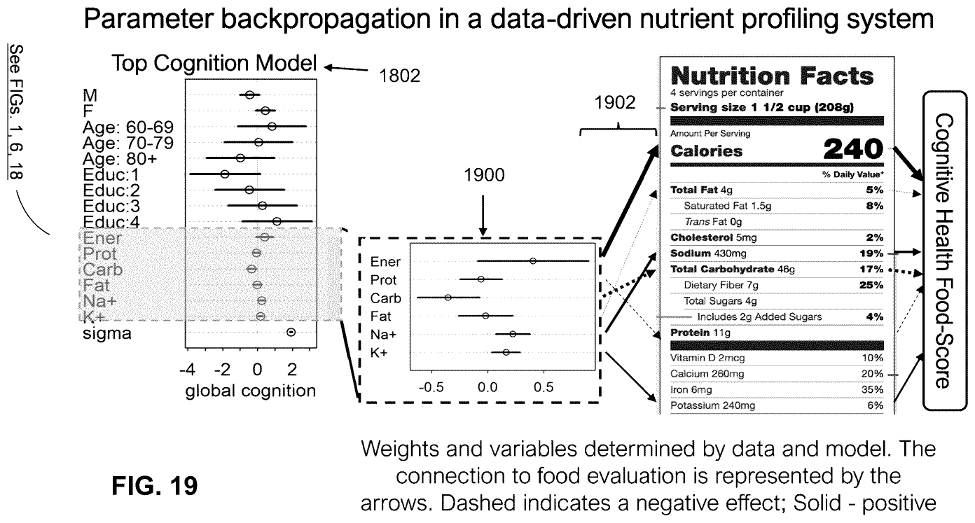

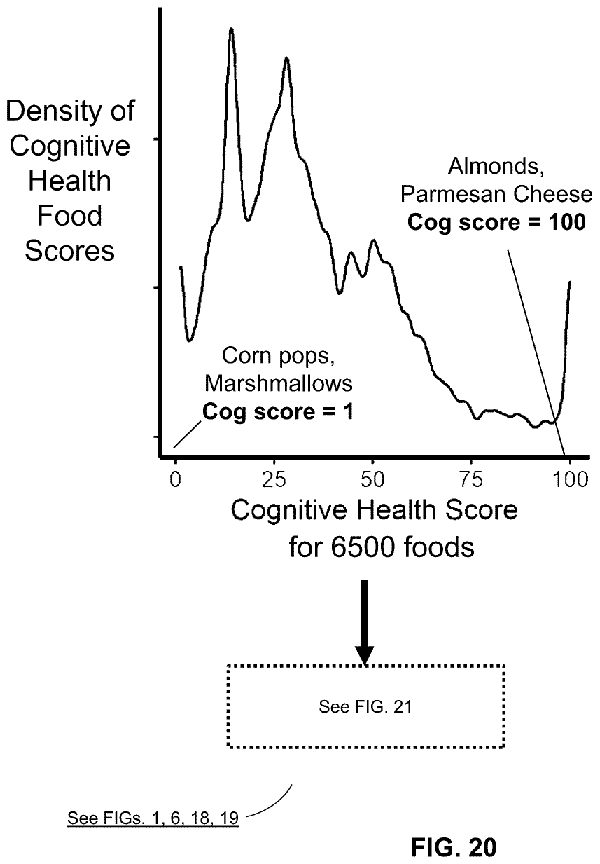

FIGS. 17-18 provide examples to contextualize model construction, evaluation, and selection/combination and FIGS. 19-20 provide examples of the process of generating food ratings and recommendations tailored to health outcomes, derived from FIGS. 6, 17, and 18. The statistical machine learning aspect of this is what is considered a model. An outcome is mathematically derived with a combination of predictor variables in a case-by-case approach considering factors such as gender, age, energy levels, protein levels, carbohydrate levels.

In FIG. 17, a particular model 1702 selected from a list of models of an example health outcome/metric 1700 known as biological age, that were identified and quantified to be a top performer using an exemplary metric. Biological age was calculated using a multivariate Mahalanobis distance from several clinical biomarkers, a published method. FIG. 18 shows this process for another outcome, global cognition 1800, which is calculated from averaging z-scores across multiple cognitive tests, another published method. FIGS. 17 and 18 represent building the models with multiple models for a certain health outcome prior to comparing them, a prerequisite before getting to the recommendations FIG. 18 or the health layer 620. The models referenced at 612 and listed in FIGS. 17 and 18 are considered statistical machine learning. Model comparisons and predictive performance are used to select and prioritize models. But in this case, the model 1702 and 1802 are selected as the best performing statistical machine learning based models for these health outcomes using Watanabe Akaike information criteria (WAIC) in 1704 and expected log posterior predictive density difference (elpd difference) in 1800 as example evaluation metrics, pulled from 614. In these examples, we also show how nutrition facts panel variables may be exclusively utilized to create a better performing model relative to existing dietary patterns or nutrient profiling systems. In total, the example models incorporate parameters such as age, sex at birth, education nutritional variables such as energy, protein, carbohydrates, sodium and potassium.

612—Predictive model construction using data: Variables considered for modeling are derived from one or many of the following categories: 1) user's preferences/belief structures, 2) available data, and/or 3) experts. These variables may be combined and modeled linearly or nonlinearly using splines, piecewise functions, general additive models, or latent variable models that are layered with respect to variables forming a network.

614—Model performance evaluation: Statistics of model performance, can include but not limited to 1) accuracy, measured through cross-validation and its estimates (e.g., elpd difference, PSIS-LOO elpd, Brier Score, AUROC, calibration, sensitivity, specificity, time-dependent AUROC, positive predictive values, negative predictive values, precision-recall curves) wherein cross validation is performed based on the prediction task (leave one out, leave one group out, K-fold, leave future out, etc. . . . ) or 2) overall fit, measured by posterior predictive distribution checks, R2, adjusted R2, calibration assessed visually or with CORP reliability diagrams, Brier scores, etc.

616—Model selection or combination from performance or existing, prior belief structure which can include any of the following: 1) select a superior model based on performance evaluation from 614, 2) take a weighted average of the models, with weights determined by predictive accuracy (or other performance metric), or to combine models using methods such as Bayesian stacking or Bayesian model averaging (BMA) or pseudo-BMA or 3) based on user-belief or preference or available data. FIGS. 17 and 18 show the selection process for one exemplary metric and preference (e.g., select the best performing model in terms of accuracy using an estimate of leave one out cross validation—elpd difference).

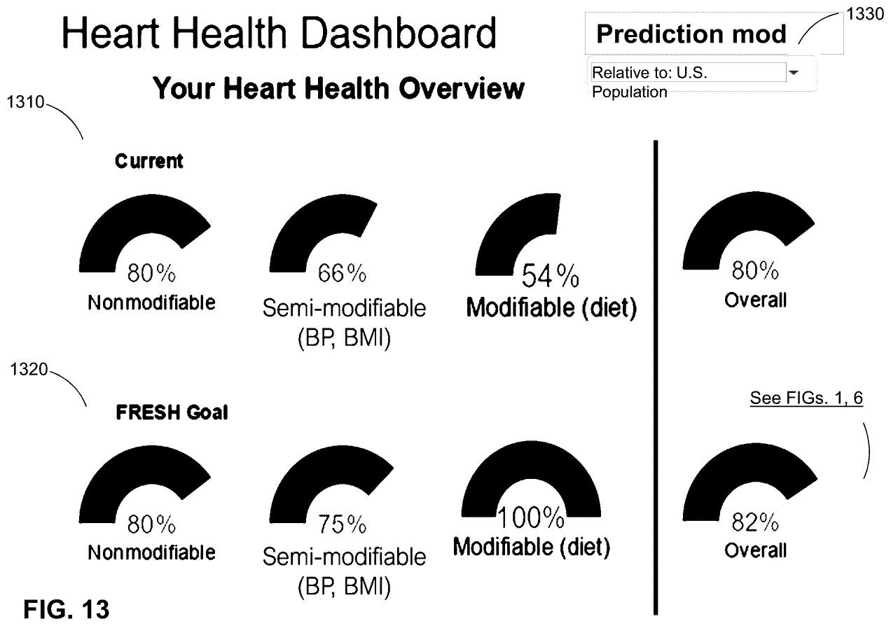

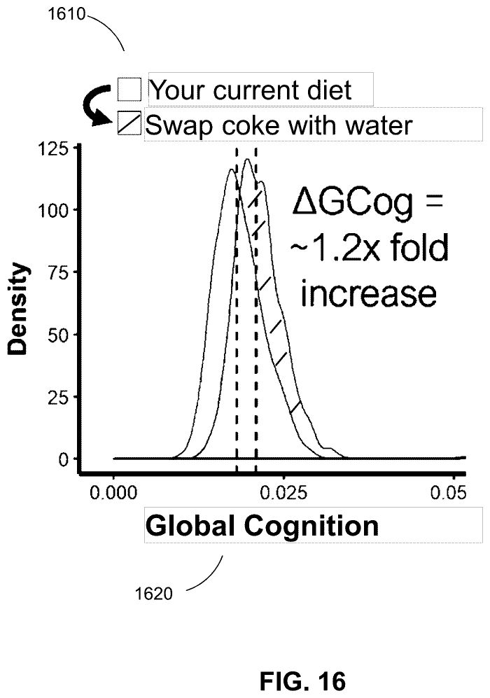

620—Risk or Health Outcome Model Outputs: User risk-factor scenario data from FIG. 5 is combined with 410 and/or 104A and/or 104B belief structure data, including preferences on health outcome preference and communication format, including but not limited to specified-time horizon risk/health event or value (e.g., 10 y), lifetime risk (probability of event or value happening for the remainder of user's life), and/or relative risk (e.g., percentile rank vs. a reference population) and processed with the predictive health model(s) from database 610. An absolute health recommendation/prediction comes out of statistical predictions from the machine/statistical-learning framework for health metrics and outcomes 110. These models can create recommendations for health for any individual or population for any combination of risk factors and their quantities, determined from the risk factor scenario generator in FIG. 5 and its prequels. Examples of these health model outputs for various lifestyle scenarios at the individual and population levels are provided in detail in FIGS. 13-16.

810—Food-level DASH scores for ˜6000 food items, an example food rating system or nutrient profiling system (NPS) that is consistent with clinical dietary patterns or index logic, are clustered or grouped by a food categorization factor (level 1 category) which can be further broken into more granularity within a hierarchically clustered food categorization system. These food scores for a NPS based on DQIs is passed into the meta-nutritional profiling system 112, forming one system among many of the expert-consensus derived nutrient profiling system database 2110 and is shown in more detail in FIG. 21.

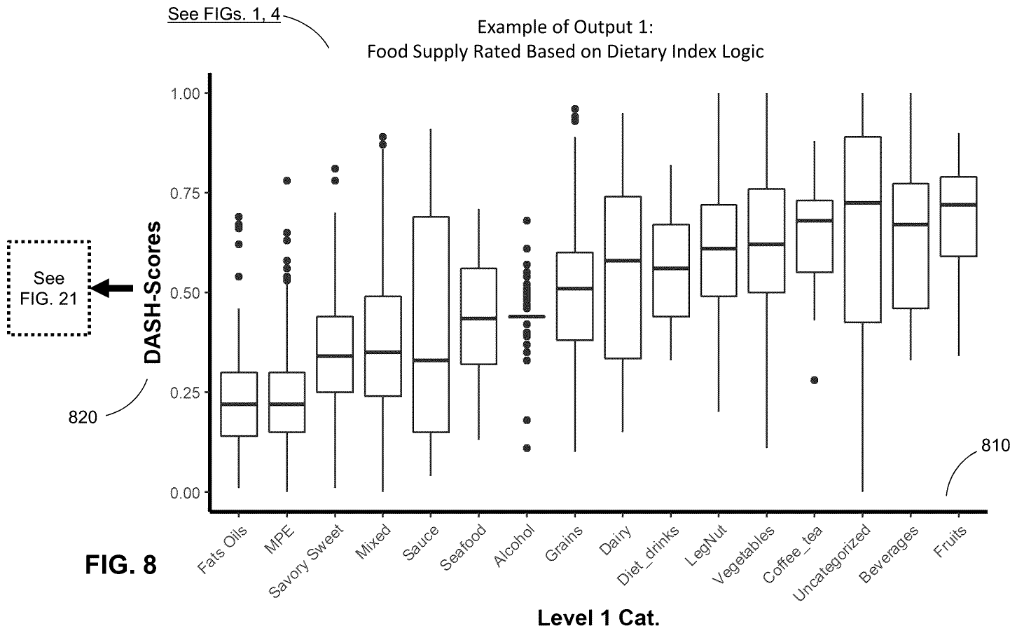

820—Shown on FIG. 8 is food ratings aligned with the DASH diet, as one example clinical diet quality index/intervention.

1310—An example schematic displaying a user's current health, as one exemplary scenario, relative to the US population as one exemplary prediction/health mode 1330.

1320—An example schematic displaying a user's future health if they changed their diet to the low-sodium DASH diet, as one exemplary scenario, relative to the US population as one exemplary prediction/health mode 1330. This scenario is shown as the most optimal according to current science, which is learned through meta-analyses of controlled feeding trials, or a network meta-analysis of existing clinical trials that compares the relative performance of all lifestyle interventions on the outcome of interest. Note in both 1310 and 1320 that health outputs are shown using speedometer charts as one graphical example, further supported by a numerical output of the users risk/health status.

1330—Health Prediction Modes: A selectable list of outputs generated from the health prediction model(s) in FIG. 6. FIG. 13 displays the individual level risk relative to the U.S. population for 2 user scenarios as a speedometer chart, with a breakdown by the variables that were parsed in the user database(s) from 104A and 104B. The overall risk or health score which combines all of these data through the use of predictive health model(s) 110 is also shown to the user.

1510—In addition to individual level risk, our engine provides population level risk factor scenarios showcasing the ability to contrast the current value scenario against the optimal scenario (for heart health) in which a population changes from their current diet to the DASH diet. These are just two examples and the framework is extendable to any scenario the user wishes to inquire about.

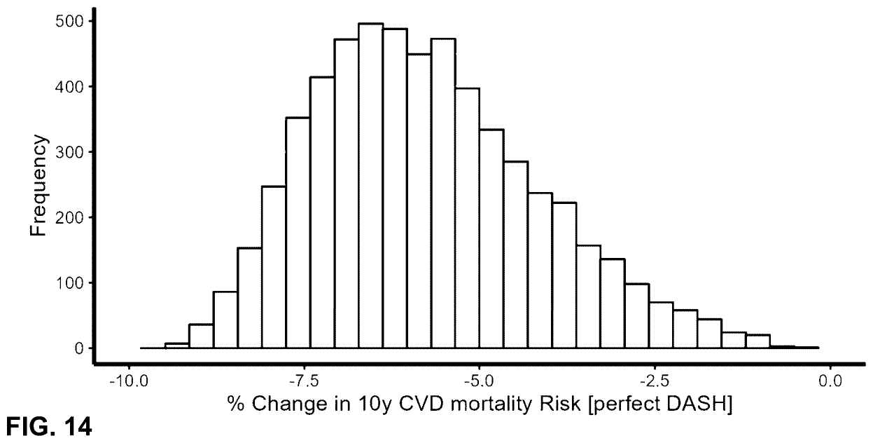

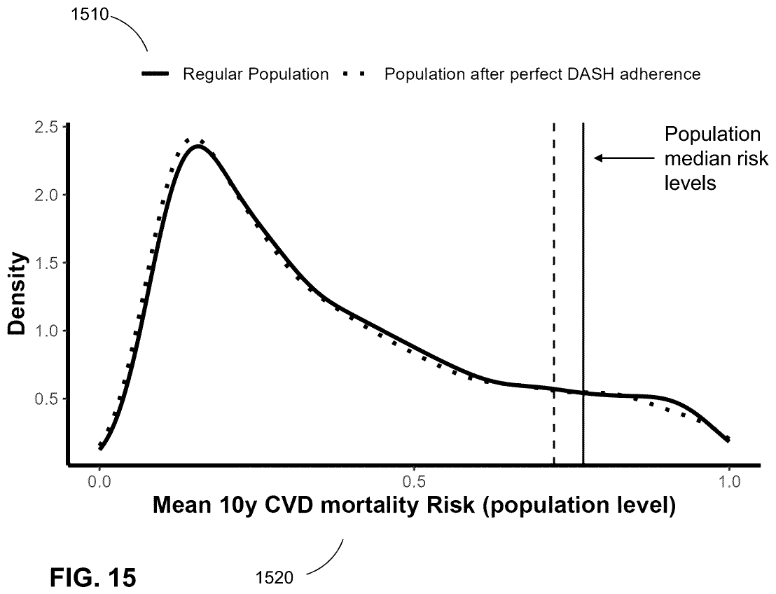

1520—The outputs are the health outcomes of these scenarios generated from the risk factor scenario generator 108 (e.g., the expected change in the absolute, population-level 10-year cardiovascular disease mortality risk) which can be displayed graphically for one or many populations using a density chart shown in FIG. 15 or a histogram. The outputs can also be represented via summary statistics of the population(s) such as the mean or median population risk with associated estimates of the uncertainty such as variance, standard deviation, and interquartile ranges. Two scenarios can be compared, and the change in relative risk at the population level can be calculated and displayed similarly, as in FIG. 14, which shows the percent change in 10y cardiovascular disease risk between the current population and this population if they adopted the DASH diet.

1610—Two individual level scenarios as examples for another health outcome (Global Cognition) showcases the multiple outcome capacity and the depth of scenarios (e.g., one food-change rather than completely changing their diet) of our recommendation engine.

1620—On example outcome, Global Cognition, is shown as an example. What this density chart shows or suggests, is that an individual who changes their current diet to one where they replace coke with water can expect a 1.2-fold increase in their cognitive health, with the width of the distribution representing the uncertainty associated with each scenario. In other words, this expected increase is not certain, and presenting the uncertainty allows the user to make more informed decisions regarding whether they want to consider enacting this behavior change.

1700—Until this point, we have discussed predictive health model outputs with little attention to how these models are constructed, evaluated, and selected 612. FIG. 17 showcases this process for one exemplary health outcome of Biological Aging.

1702—A top performing model is highlighted, who's components in this case are factors from user databases (e.g., age, sex, energy, protein, carbohydrate, fat, sodium, and potassium), which was selected on the basis of 1704, as one example metric from a database of performance metrics 614.

1704—A graphical representation of one the Watanabe Akaike Information Criteria, a metric of model accuracy and predictive performance, is used to compare all of the constructed models for this health outcome.

1800—Similarly, another health outcome may be modeled in a specific dataset or groups of datasets, where specific dietary scenarios or rating systems may be compared for their capacity to predict a health outcome. The details in our Cognition example in terms of the dataset and potential models being compared are shown in FIG. 18.

1802—The expected log posterior predictive density difference, one exemplary performance metric from 614, was utilized to select the best performing model for cognition. This model is utilized to showcase the data-driven nutrient profiling system that is tailored to this health outcome in FIGS. 19 and 20. An inventive aspect of a data-driven, health outcome specific nutrient profiling system is parameter backpropagation through food composition databases. In certain situations, a subset of variables that are restricted to the nutrition facts panels may be utilized to offer maximum scalability to evaluate the entire food supply.

1900—In parameter backpropagation, the nutritional parameters of the top performing model, and their uncertainty, are utilized to rate the healthfulness of the food. The relative weights and directions (e.g., either positive or negative) are learned through the data and the model from earlier steps.

1902—Parameter weights/directions are shown as arrows, with the magnitude reflected in the weight of the arrow, and the direction shown by either a solid (for positive) or dotted (negative) line, as example graphical representations.

2110—A database of expert-derived food rating systems (i.e., nutrient profiling systems (NPS)). The individual NPS may be derived from DQIs, as explained and showcased in FIG. 8 and associated subcomponents, or may be derived from literature which contains over 200 individual NPS.

2120—A database of data-driven, health outcome specific food rating systems (i.e., nutrient profiling systems (NPS) tailored to cognition or biological aging). Examples are shown in FIGS. 19-20.

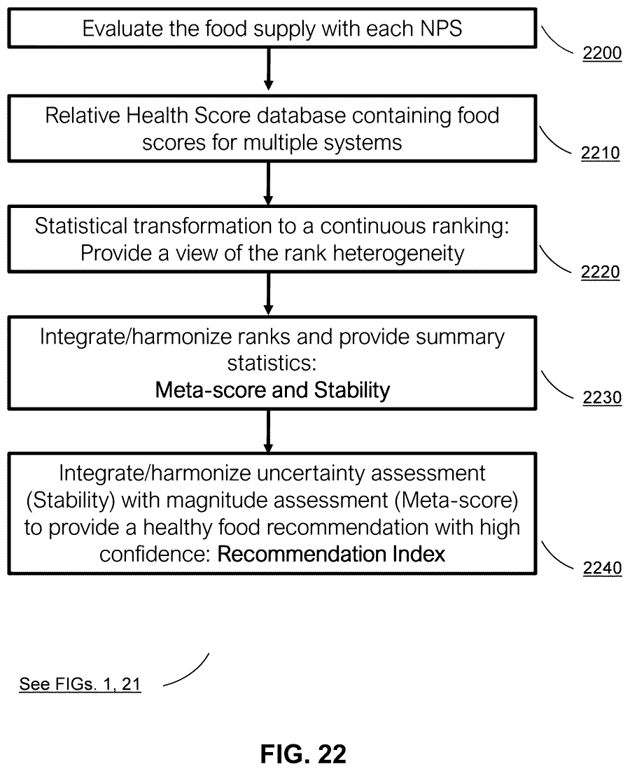

2130—A healthfulness evaluation harmonization engine for human health takes multiple food rating systems from 2110 and 2120 and turns them into a consensus evaluation of health through a process shown graphically in FIG. 22, providing a consensus evaluation of the healthfulness of foods given multiple systems and the user preferences for these systems.

2132—Planetary healthfulness is optionally assessed by layering in sustainability metric data from life cycle analysis databases at the food level 2140.

2134—Cost-effectiveness is optionally assessed as a final step by layering in food-level economic data from a price and affordability database 2150.

2140—Life cycle analysis databases may contain assessments of a food's planetary healthfulness via metrics such as greenhouse gas emissions, land use, water scarcity, eutrophication potential, and ecosystem diversity.

2200—As relative health assessments, in order to understand how healthy a certain food is, the entire food supply (or a representative sample of it) needs to be assessed with a food rating system. Thus, the first step of the meta-NPS is to assess this food supply with each NPS, which is then passed into a database containing the food-level relative healthfulness evaluation for each system 2210.

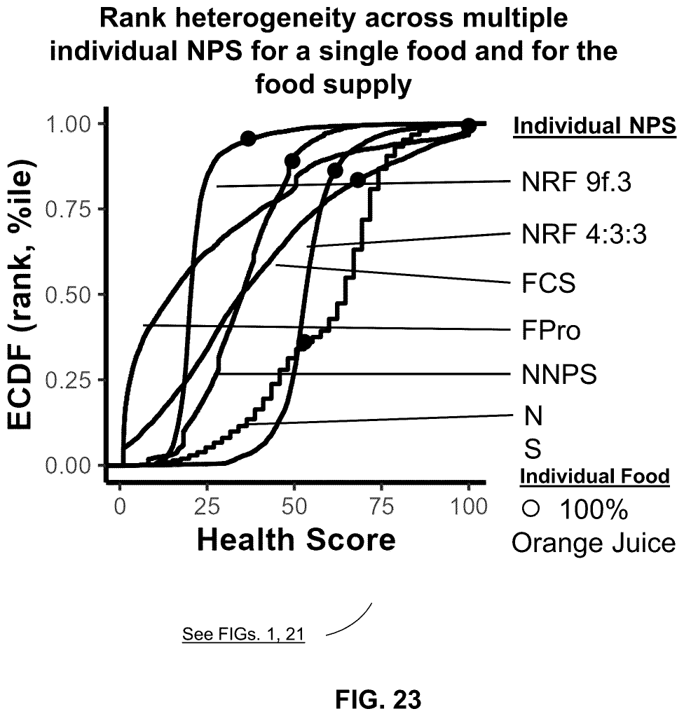

2220—In the case that the scores from various systems are on different numerical scales with different health interpretations, statistical transformation/harmonization is required to create a continuous ranking system. The empirical cumulative distribution function (ECDF) may be utilized as one example rank-based transformation that yields continuous percentile ranks. At this point, food ratings across systems may be viewed and compared to show the degree of cross-system heterogeneity as in FIG. 23. The degree to which each system scores the food supply can be evaluated with summary statistics of the empirical cumulative distribution curves or with the density charts (not shown). In addition, food level healthfulness variation may be overlaid on these charts to more clearly showcase single food items.

2230—With multiple variable ranks for a given food, the next step of the meta-NPS is to begin harmonizing these ranks and providing summary statistics. Any central tendency statistical measure (e.g., mean, median, mode) can be utilized to create the Meta-score whereas any variance/uncertainty estimator (e.g., standard deviation, variance, coefficient of variation, interquartile range, and/or their inverses) may be utilized to create the Stability score. Together these two health score properties-Meta-score and Stability-represent the consensus healthfulness rank of a given food and the relative degree of confidence in those rankings, respectively.

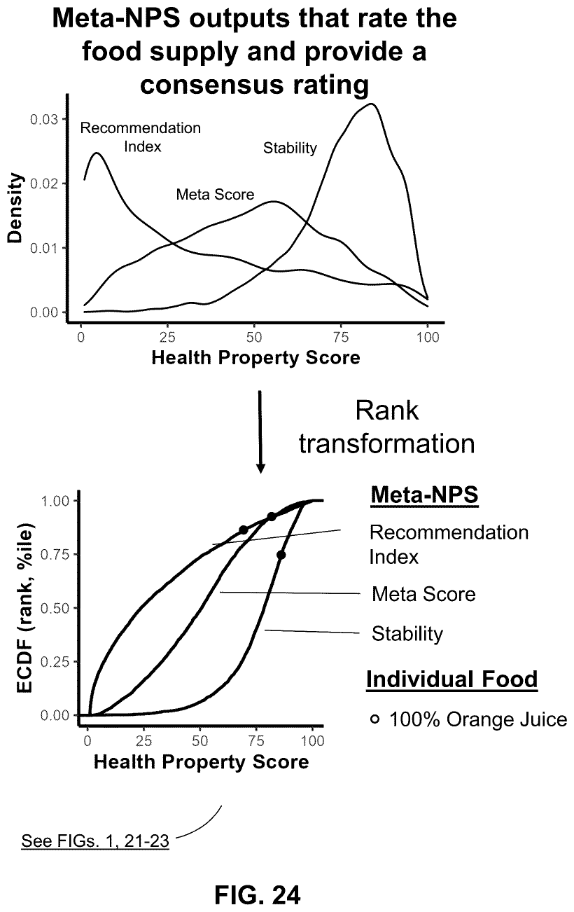

2240—As a final step to provide a single metric that summarizes the harmonization process, a Recommendation Index is created through mathematically/statistically combining Meta-score and Stability (e.g., multiplying, addition, subtraction, etc. . . . ). The Recommendation Index provides an intuitive property for being a metric that identifies healthy foods-we want to find foods that are the healthiest (high Meta-score) with high confidence (high Stability). All of these processes are shown in FIG. 24. The resulting graphs or numerical ratings or any other representation of the consensus evaluation for cost-effective human and planetary health foods are then projected back to the user on a website, mobile device, or other dashboard 114.

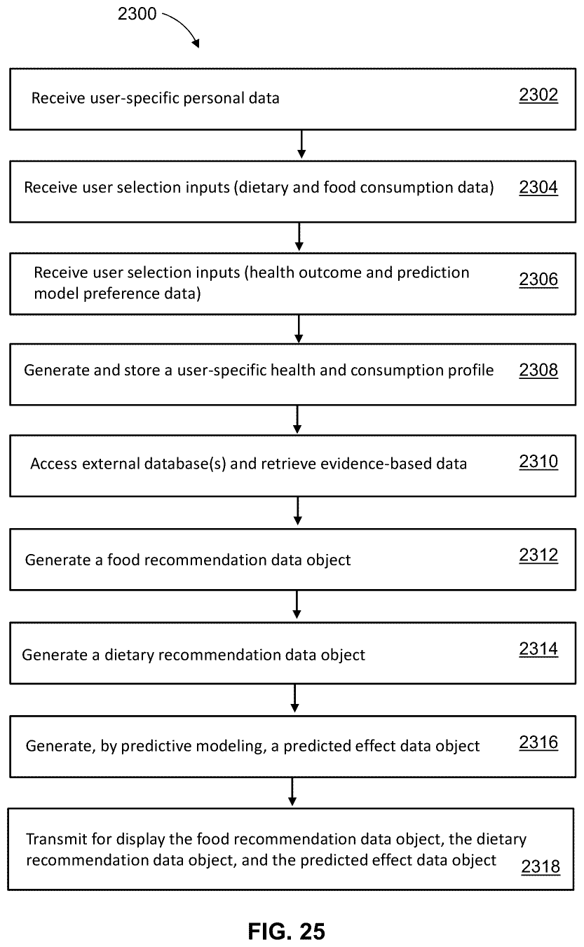

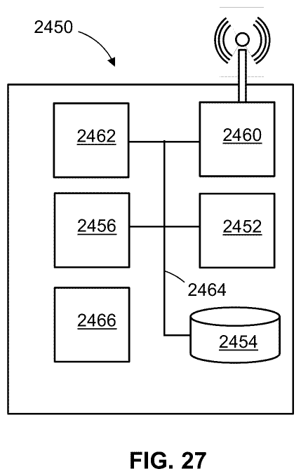

In some embodiments, a method is provided for a computing system to provide a food and dietary recommendation and corresponding predicted effects profile. FIG. 25 diagrammatically represents such a method, referenced as method 2300, contemplated as implemented by a computing system that includes one or more processor, and at least one memory device storing computer-readable instructions. The method 2300 (FIG. 25) can for example be implemented by use of any and all of user-side user devices 2422, 2424, 2426 and provider-side computing devices 2432 and 2434, in cooperation or in communication with each other, with further reference as well to FIG. 27 and descriptions thereof below. The one or more processor is configured to execute the computer-readable instructions thereby causing the processor to performs steps of the referenced method 2300.

The method 2300 includes: a step to receive (2302), from a user device, user-specific personal data comprising user-specific biological metrics; a step (2304) to receive, from the user device, user selection inputs comprising user-specific dietary and food consumption data; and a step (2306) to receive, from the user device, user selection inputs comprising user-specific health outcome and prediction model preference data.

The method 2300 further includes: a step (2308) to automatically generate and store, in a system-internal database, a user-specific health and consumption profile based at least on the received user-specific personal data and user selection inputs; and a step (2310) to access at least one external database and retrieve therefrom evidence-based data associated with at least portions of the user-specific health and consumption profile.

The method 2300 further includes: a step (2312) to generate a food recommendation data object based at least in part on the user-specific biological metrics and retrieved evidence-based data; a step (2314) to generate a dietary recommendation data object based at least in part on the user-specific biological metrics and retrieved evidence-based data; and a step (2316) to generate, by predictive modeling, a predicted effect data object based at least in part on the dietary recommendation data object and the user-specific biological metrics.

The method 2300 further includes: a step (2318) to transmit for display, at least in part, on the user device: the food recommendation data object; the dietary recommendation data object; and the predicted effect data object.

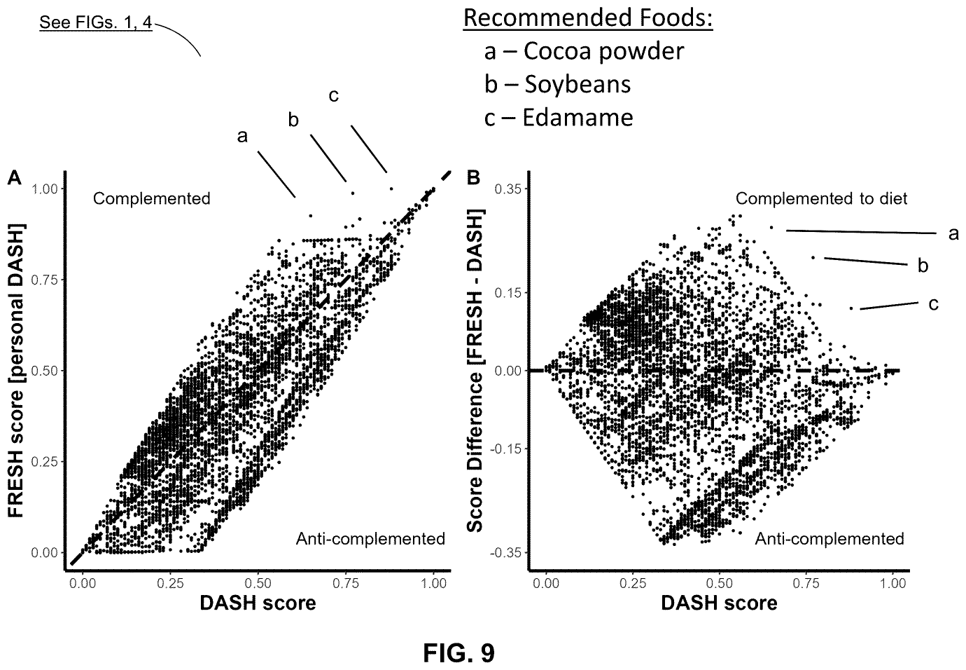

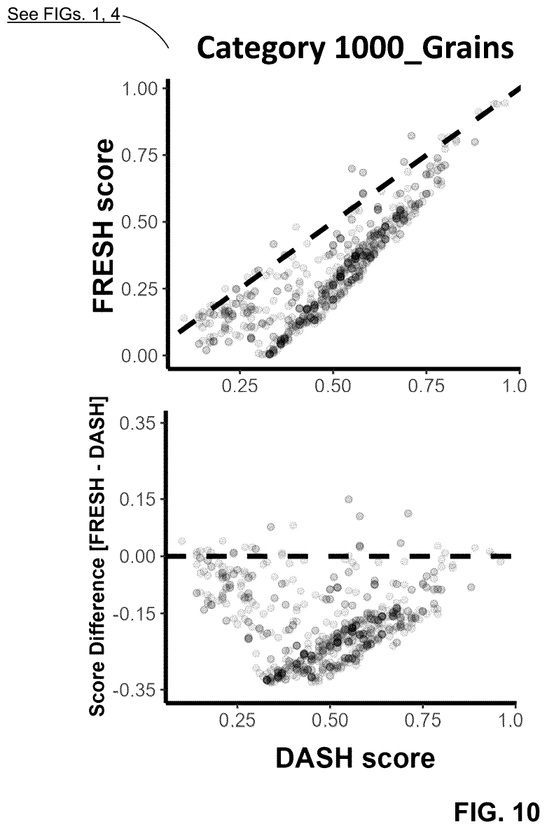

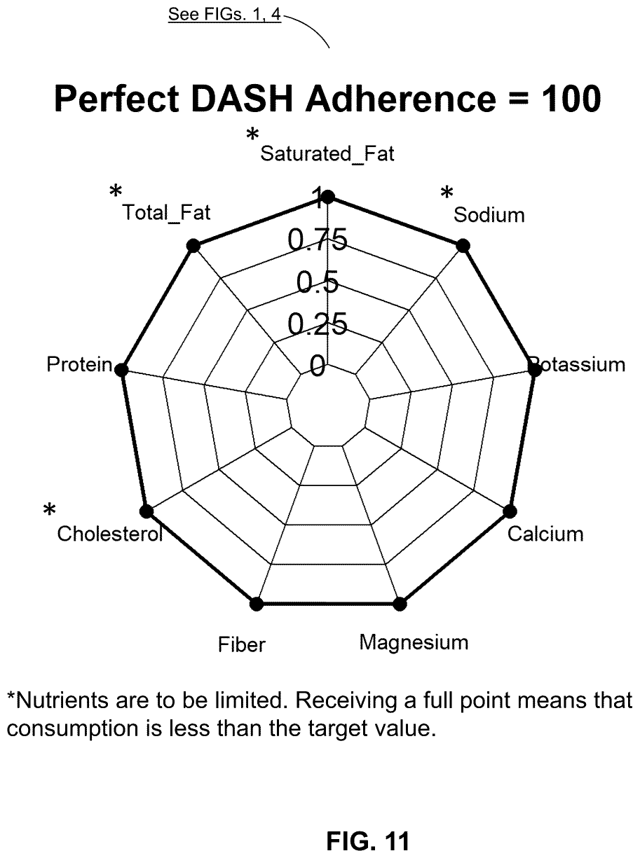

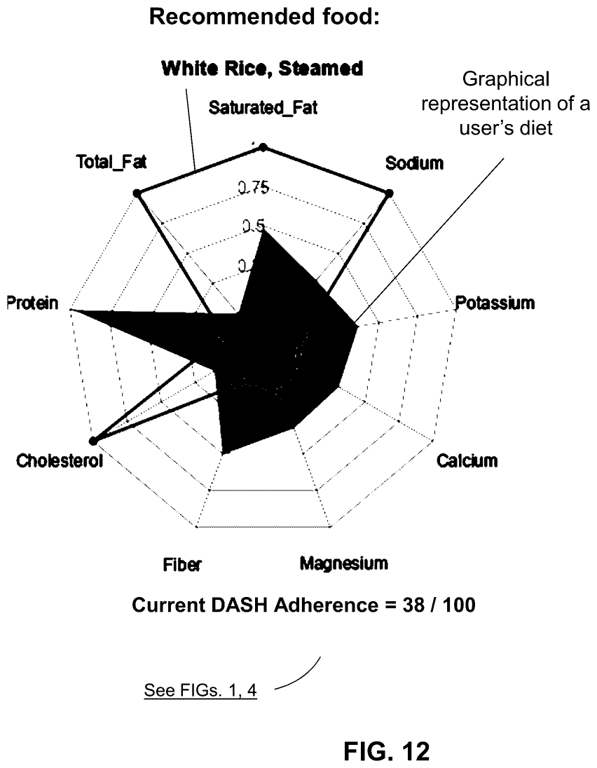

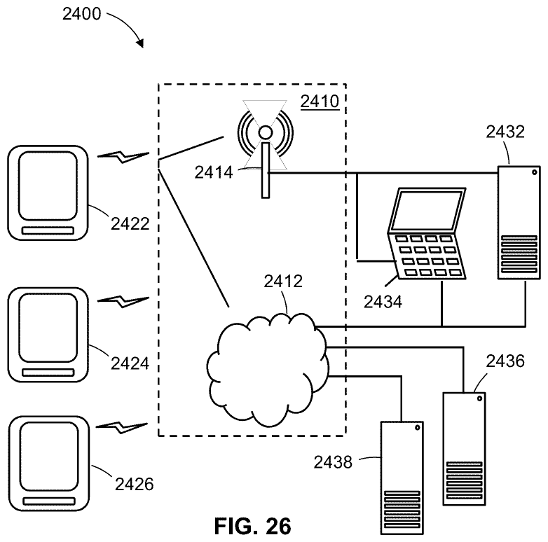

In some embodiments, the methods, systems, and devices described or implied herein are implemented on or as a single user device, such a smart phone. In other embodiments, the methods, systems, and devices described or implied herein are accessed through user devices and further implemented vie service-provider side computing, such as by a provider's computing device or cloud-computing service. Reference numbers in the following descriptions with reference to FIGS. 25 and 26 are separate from any similar reference numbers in the above descriptions with reference to FIG. 1-24, without ambiguity, in that like numbers in the above do not refer to same or similar features as like numbers in the below.

FIG. 26 is diagrammatic representation of a system 2400 including a network 2410, which may include the internet 2412 and a local area network 2414 in any combination. The computing devices 2422, 2424, and 2426, representing user devices such as the smart phones of subscribers, may communicate via the network 2410 or directly by wired or wireless connection. In at least some embodiments, the various phones or user devices represented in, for example, FIG. 1, FIG. 4, and FIG. 6 are exemplified as computing devices 2422, 2424, 2426 in FIG. 25.

On the service provider side, computing devices 2432 and 2434 are illustrated. The system 2400 is capable of executing any or all aspects of software and/or application components presented herein on computing devices, be that client-side user devices 2422, 2424, 2426 and/or provider side computing devices 2432 and 2434, in cooperation or in communication with each other. Computing devices 2436 and 2438 represent, for example, data sources and/or other service providers.

Methods and systems described herein may be implemented using hardware or a combination of software and hardware, either in a dedicated computing device, or integrated into another entity, or distributed across multiple entities or computing devices, including user devices 2422, 2424, 2426 and/or provider-side computing devices 2432 and 2434.

By way of example, and not limitation, each computing device 2432, 2434, 2436 and 2438 is intended to represent any form of digital computer, including a mobile device, a server, a blade server, a mainframe, a mobile phone, a personal digital assistant (PDA), a smart phone, a desktop computer, a netbook computer, a tablet computer, a workstation, a laptop, and any other computing device. The components shown here, their connections and relationships, and their functions, are meant to be exemplary only, and are not meant to limit implementations of the invention expressly described and/or claimed.

FIG. 27 is a diagrammatic representation of a computing device 2450, which can represent any of the client-side user devices 2422, 2424, 2426 and/or provider-side computing devices 2432 and 2434 of FIG. 27. The computing device 2450 includes components such as a processor 2452, a storage device or memory 2454. A communications controller 2456 facilitates data input and output to an interface 2460 graphically represented as radio but more generally representing or including any wired or wireless connection device. Input and output devices 2462 such as a screen, a keyboard, a speaker, and/or other buttons facilitate interface with and use by a user. Examples of input and output devices 2462 include, but are not limited to, alphanumeric input devices, mice, electronic styluses, display units, touch screens, signal generation devices (e.g., speakers) or printers, scanners, and microphones. A system bus 2464 or other link interconnects the components of the computing device 2450. A power supply 2466, which may be a battery or voltage device plugged into a wall or other outlet, powers the computing device 2450 and its onboard components.

By way of example, and not limitation, the processor 2452 may be a general-purpose microprocessor such as a central processing unit (CPU), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and other manipulations of information.

The storage device or memory 2454 may include, but is not limited to: volatile and non-volatile media such as cache, RAM, ROM, EPROM, EEPROM, FLASH memory or other solid state memory technology, disks or discs or other optical or magnetic storage devices, or any other medium that can be used to store computer readable instructions and which can be accessed by the processor 2452. In at least one example, the storage device or memory 2454 represents a non-transitory medium upon which computer readable instructions are stored, and when such instructed are executed by the processor 2452, the computing device 2450 implements, in whole or in part, methods described herein by serving as a system or device as described herein or a part thereof.

Inventive aspects are numbered in the following descriptions, some of which includes parenthetic content representing examples, explanations, context, and options.

Aspect 1—A system configured to take a plurality of user inputs from various domains, reference this data to external knowledge databases (an evidence-base), process all the variables/information, and provide back to the user: i) food and diet recommendations consistent with guidelines, user consumption patterns, and multiple food rating systems either expert-derived for general health and/or data-driven and specific to a health condition; and ii) predicted effects on one or many health outcomes, and their uncertainty, for either one or a multitude of health and lifestyle scenarios (e.g., comparing a user's current health status with that of a future health status if the user adopted a clinically proven lifestyle intervention).

Aspect 2—The system of Aspect 1, the system further configured to providing uncertainty assessment, consistency with existing dietary guidelines, compatibility with belief structures, and/or integration with predictive health models.

Aspect 3—A meta-nutritional profiling system to provide food and diet recommendations to optimize individual and population health, the system including at least: a first sub-system which incorporates belief structures, external databases and/or observations, user databases, and performs predictive modeling for one or many health outcomes; and a second sub-system which incorporates existing, expert derived food scores and/or ranks; wherein the combination of these two subsystems generates a meta-system with resulting Meta-Score, Stability, and Recommendation Index measures (e.g., adaptable food recommendations for health, given certain inputs from the user reflecting their beliefs).

Aspect 4—A system to provide a consensus healthfulness evaluation of foods through statistically/mathematically combining multiple nutrient profile models (food rating systems), which may be derived from experts (published models), from dietary quality index logic (see Aspect 1), or from data-driven modeling of diet and/or food(s) relationship to health parameters (see Aspect 1).

Aspect 5—The system of Aspect 1, wherein the system further accounts for cost and environmental effects, creating a food recommendation system for environmental sustainability and health (FRESH).

Aspect 6—A method, framework, and/or system configured to implement the method, to identify maximally predictive diets and lifestyles from data, consistent with the belief structures of the individual, the method including at least: incorporating measured data from lifestyle factors, biological samples/data, health outcomes of interest, and environmental effects on said outcomes; and incorporating data on the construction, fitting, and performance evaluation of multiple prediction models for the outcomes of interest.

Aspect 7—The method framework, and/or system of Aspect 3, wherein belief structures are incorporated at least three distinct locations or implementations: the variables used in predictive modeling, and their relative effects, for example, the individual may specify that, exercise is twice more important than diet, and another biological parameter (e.g., white blood cell number) is not relevant; another example may be a user that prefers modifying their physical activity over diet to maximize their health for a given set of outcomes; their goals or preferences in achieving health outcomes, for example, the user may select to prioritize optimizing reducing cardiovascular disease risk at the cost of increased dementia risk; and how much they value their own health vs. a separate population vs. a planet/environment.

Aspect 8—The method framework, and/or system of any preceding numbered Aspect, wherein all above variables are input into model structures, that are fit and assessed with predictive statistical algorithms.

Aspect 9—The method framework, and/or system of any preceding numbered Aspect, wherein uncertainty from all above data sources, and belief structures, may be incorporated to inform the predictions, rendering a more precise and accurate prediction required for optimal decision making.

Aspect 10—The method framework, and/or system of any preceding numbered Aspect, wherein said statistical models representing the belief structures of the individual and the given data/observations are compared on performance measures such as cross-validation, elpd difference, log-likelihood, WAIC scores, ROC, adjusted R2, posterior predictive checks, etc.

Aspect 11—The method framework, and/or system of any preceding numbered Aspect, wherein the best predictive model (or subset/combination of models) is selected for making predictions of diet's effect on the health outcome.

Aspect 12—The method framework, and/or system of any preceding numbered Aspect, wherein diets and/or food(s) and their health effects are simulated and/or calculated through (back) propagating the selected model(s) parameters through a food composition database, generating both absolute and relative health-effect predictions and food scorings.

Aspect 13—The method framework, and/or system of any preceding numbered Aspect, wherein said propagation is performed through posterior predictive distributions from selected models or from summary statistics of the parameters.

Aspect 14—The method framework, and/or system of any preceding numbered Aspect, wherein at least one instance focuses solely on use of variables within the nutrition facts panel for dietary variables of interest.

Aspect 15—The method framework, and/or system of any preceding numbered Aspect, wherein predicted effects for the consumption of a diet or food for an individual or a population may be delivered to the user on an absolute or relative scale.

Aspect 16—A method, framework, and/or system to aggregate food scoring systems and communicate uncertainty in resulting scores/recommendations, wherein the method, framework, and/or system: incorporates both data-driven, and/or expert-consensus based scoring systems; incorporates the individual's (e.g., person, institution, organization, community) relative beliefs, or emphasis, on which family of systems they trust, such as they may value the scores for the data-driven systems by X % (for example 40%) and experts by a complementary percentage 100%−X % (for example 100%−40%=60%), and these beliefs represented as numbers, are translated into a personalized, hybrid data/expert-driven food recommendation system for that individual; and incorporates and communicates relative uncertainty associated from aggregating scoring systems at a meta-level.

Aspect 17—A method, framework, and/or system to aggregate food-recommendation systems for health with food-recommendation for economic and environmental sustainability, wherein the method, framework, and/or system takes the scoring systems from previous two systems, and overlays the food/beverage product or diet scores with cost/affordability databases as well as life-cycle inventory databases, wherein the food recommendation that takes into account health and environmental sustainability is communicated through scores or ratings, either numerically or graphically.

Aspect 18—A method, framework, and/or system whereby the consensus food ratings is utilized for the applications of one or more of: diet and food consumption; food reformulation and front-of-pack labeling; investment decisions; and engineering design criteria for bioengineered food products.

Aspect 19—A method or system configured to identify and recommend foods that are complemented and anti-complemented to a user's diet quality (or a plurality of users' diets).

Aspect 20—A method or system configured to communicate a food's healthfulness and its multivariate complexity by overlaying its characteristics on top the user's diet quality, assessed using those same characteristics or variables.

Aspect 21—The system of Aspect 20, further configured in the form of a spider chart or a bar graph containing all of the variables needed to be consistent with each other and with dietary guideline logic.

Aspect 22—A method or system configured to communicate the heterogeneity in the definition of healthfulness or a healthy food, using multiple rating systems or guidelines all designed to rank the healthfulness of foods.

Aspect 23—The method framework, and/or system of any preceding numbered Aspect, wherein predictive health model(s) are used to select combinations of variables to generate and define a healthy range of consumption for a given diet, food, or nutrient, or any combination thereof.

Aspect 24—The method framework, and/or system of any preceding numbered Aspect, wherein predictive health model(s) are used to select combinations of variables to generate and define a healthy, optimal ranges for semi-modifiable physiological factors individually or in combination for a given outcome or collection of health outcomes.

Aspect 25—The method framework, and/or system of any preceding numbered Aspect, wherein predictive health model(s) are used to understand and estimate health disparities defined by combinations of non-modifiable factors (e.g., age, sex, race/ethnicity, education, and geolocation).

Aspect 26—The method framework, and/or system of any preceding numbered Aspect, wherein predictive health model(s) are used to quantitatively compare and prioritize multiple competing lifestyle interventions in terms of their capacity to affect a future health state, to be further evaluated with randomized controlled clinical trials.

Embodiments of the present invention are described above and herein with reference to illustrations and/or block diagrams of systems, devices, and methods, each to be understood such that each function described or implied can be implemented by computer program instructions. These computer program instructions in some implementation are to be provided to a processor of a general use computer, special use computer, or other programmable data processing machine, such that when the instructions executed, the functions, acts, calculation, inputs, outputs, and displays described, illustrated, and/or implied above are implemented.

These computer program instructions can be stored in a non-transitory computer-readable memory that can direct a computer or other programmable data processing apparatus to function in a particular manner, such that the instructions stored in the computer readable memory produce an article of manufacture including instructions, which implement the function/act described, illustrated, and/or implied.

The computer program instructions may be loaded onto a computer or other programmable data processing apparatus to cause a series of operational steps to be performed on the computer or other programmable apparatus to produce a computer implemented process such that the instructions, when executed, provide steps for implementing the functions/acts described, illustrated, and/or implied. Computer program implemented steps or acts may be combined with operator or human implemented steps or acts in order to carry out embodiments of the invention.

Where devices, systems, and their functions are described herein, corresponding methods and flowcharts thereof are described as well, whether expressly provided in the drawings or implied as would be understood by one of ordinary skill in the art to which the subjects matters of these descriptions pertain.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

What is claimed is:

1. A system for providing a food and/or dietary recommendation and a predicted effects profile, the system comprising:
at least one processor;
a communication interface communicatively coupled to the at least one processor; and
a memory device storing executable code that, when executed, causes the processor to:
receive, from a user device, user-specific personal data comprising user-specific biological metrics;
receive, from the user device, user selection inputs comprising user-specific dietary and food consumption data;
receive, from the user device, user selection inputs comprising user-specific health outcome and prediction model preference data;
automatically generate and store, in a system-internal database, a user-specific health and consumption profile based at least on the user-specific personal data and user selection inputs;
access at least one external database and retrieve therefrom evidence-based data associated with at least portions of the user-specific health and consumption profile;
generate a food recommendation data object based at least in part on the user-specific social, environmental, and biological metrics and retrieved evidence-based data;
generate a food recommendation data object aligned with existing dietary guidelines or patterns based at least in part on retrieved evidence-based data containing multiple dietary quality metrics and/or indices and their listed scoring components;
generate a food recommendation data object aligned with expert's food healthfulness opinions based at least in part on retrieved evidence-based data containing multiple food quality metrics and/or indices and their listed scoring components identified and pooled from the scientific literature on nutritional profiling systems/algorithms, including an assessment of the consensus food rating across multiple expert-driven algorithms;
generate a dietary recommendation data object based at least in part on the user-specific biological metrics and retrieved evidence-based data;
generate a dietary recommendation data object based on the retrieved evidence-based data containing clinical effect sizes for health outcomes stratified by dietary quality index/measure/guideline;
generate a multi-dimensional, visual dietary recommendation data object known as a spider or radar chart based on the user-specific dietary quality and retrieved evidence-based data containing multiple dietary quality metrics and/or indices;
generate, by predictive modeling, a predicted effect data object based at least in part on the dietary recommendation data object and the user-specific biological metrics that are not blood glucose;
generate, by either predictive modeling and/or via a prior belief elicitation method, an uncertainty data object based on a survey question and/or framework coded into a prior distribution of effect size or relative variable importance, the degree of missingness in the food and or diet data, the variation/distribution in prediction results across multiple predictive models, the distribution of predictions resulting from the same model, variation in the target prediction task, and/or variation in individuals and subgroups;
generate, by an expert-belief elicitation method using nutritional profiling algorithms that reflect various expert beliefs on food healthfulness, an uncertainty data object based the variation/distribution in prediction results across multiple predictive models, the distribution of predictions resulting from the same model, variation in the target prediction task, and/or variation in individuals and subgroups;
generate, by either predictive modeling and/or via a prior belief elicitation method, a quality data object based on a survey question coded into a prior distribution of effect size or importance, a measure to quantify the degree of accuracy of the prediction, the variation in predicted results across multiple predictive models ranked by their predictive accuracy, and/or based on variation in accuracy among individuals and subgroups; and
transmit for display, at least in part, on the user device:
the food recommendation data object;
the dietary recommendation data object
the predicted effect data object;
the uncertainty data object; and
the quality data object.

2. The system of claim 1, wherein the predictive modeling is conducted by back propagating model parameters selected from the top, most accurate model(s) identified through a process of evaluating multiple models on the basis of their predictive accuracy, and its uncertainty, via variations on methods such as cross-validation, such as leave-one-out, leave-future-out, leave-group-out, 5-fold, 10-fold, and quantified via metrics such as expected log posterior predictive density, Watanabe Akaike information criteria, root mean squared error, R2, adjusted R2, Brier scores, calibration assessed with stable reliability diagrams and CORP, net reclassification index, etc simultaneously through a food composition database and generating both absolute and relative health-effect predictions and food scorings.

3. The system of claim 2, wherein said back propagating is performed through posterior predictive distributions from selected model(s), thus including an assessment of both within and between model uncertainty, or from summary statistics of the model parameters.

4. The system of claim 1, wherein at least one predictive health model is used to quantitatively compare and prioritize multiple competing lifestyle interventions in terms of their capacity to affect a future health state.

5. The system of claim 1, wherein multiple food scoring systems are aggregated either from expert opinions represented as nutritional profiling systems from a published scientific article, from a data-driven modeling approach of claim 2, and/or a hybrid expert-data driven food scoring system, to generate the food recommendation data object comprised of an expert consensus evaluation, which can be defined by created a summary distribution of health scores for any given food.

6. The system of claim 1, wherein a respective uncertainty is transmitted with each of the food recommendation data object—expressed as a statistical distribution and summary statistics derived thereof representing variations in food quality or healthfulness across multiple food health or nutrition profiling systems, the dietary recommendation data object—expressed as a statistical distribution and summary statistics derived thereof representing variations in diet quality across multiple diet quality indices/patterns/metrics—, and the predicted effect data object—expressed as a statistical distribution and summary statistics derived thereof representing variations in expected effects.

7. The system of claim 1, wherein to generate the food recommendation data object, the processor uses data-driven scoring systems, and/or expert-consensus based scoring systems, which are defined by aggregating individual expert opinions about what constitutes a healthy food codified into a nutritional profiling algorithm, by combining them into a meta-nutritional profiling system.

8. The system of claim 1, wherein to generate the food recommendation data object, the dietary recommendation data object, and the predicted effect data object, the processor utilizes user-specific relative beliefs.

9. A method for a computing system to provide a food and/or dietary recommendation and a predicted effects profile, the computing system including:
at least one processor;
a communication interface communicatively coupled to the at least one processor; and
a memory device storing executable code that, the method comprising, when the executable code is executed, the at least one processor causing the computing system to:
receive, from a user device, user-specific personal data comprising user-specific biological metrics;
receive, from the user device, user selection inputs comprising user-specific dietary and food consumption data;
receive, from the user device, user selection inputs comprising user-specific health outcome and prediction model preference data;
automatically generate and store, in a system-internal database, a user-specific health and consumption profile based at least on the user-specific personal data and user selection inputs;
access at least one external database and retrieve therefrom evidence-based data associated with at least portions of the user-specific health and consumption profile;
generate a food recommendation data object based at least in part on the user-specific social, environmental, and biological metrics and retrieved evidence-based data;
generate a food recommendation data object aligned with existing dietary guidelines or patterns based at least in part on retrieved evidence-based data containing multiple dietary quality metrics and/or indices and their listed scoring components;
generate a food recommendation data object aligned with expert's food healthfulness opinions based at least in part on retrieved evidence-based data containing multiple food quality metrics and/or indices and their listed scoring components identified and pooled from the scientific literature on nutritional profiling systems/algorithms, including an assessment of the consensus food rating across multiple expert-driven algorithms;
generate a dietary recommendation data object based at least in part on the user-specific biological metrics and retrieved evidence-based data;
generate a dietary recommendation data object based on the retrieved evidence-based data containing clinical effect sizes for health outcomes stratified by dietary quality index/measure/guideline;
generate a multi-dimensional, visual dietary recommendation data object known as a spider or radar chart based on the user-specific dietary quality and retrieved evidence-based data containing multiple dietary quality metrics and/or indices;
generate, by predictive modeling, a predicted effect data object based at least in part on the dietary recommendation data object and the user-specific biological metrics that are not blood glucose;
generate, by either predictive modeling and/or via a prior belief elicitation method, an uncertainty data object based on a survey question and/or framework coded into a prior distribution of effect size or relative variable importance, the degree of missingness in the food and or diet data, the variation/distribution in prediction results across multiple predictive models, the distribution of predictions resulting from the same model, variation in the target prediction task, and/or variation in individuals and subgroups;
generate, by an expert-belief elicitation method using nutritional profiling algorithms that reflect various expert beliefs on food healthfulness, an uncertainty data object based the variation/distribution in prediction results across multiple predictive models, the distribution of predictions resulting from the same model, variation in the target prediction task, and/or variation in individuals and subgroups;
generate, by either predictive modeling and/or via a prior belief elicitation method, a quality data object based on a survey question coded into a prior distribution of effect size or importance, a measure to quantify the degree of accuracy of the prediction, the variation in predicted results across multiple predictive models ranked by their predictive accuracy, and/or based on variation in accuracy among individuals and subgroups; and
transmit for display, at least in part, on the user device:
the food recommendation data object;
the dietary recommendation data object
the predicted effect data object;
the uncertainty data object; and
the quality data object.

10. The method of claim 9, wherein the predictive modeling is conducted by back propagating model parameters selected from the top, most accurate model(s) identified through a process of evaluating multiple models on the basis of their predictive accuracy, and its uncertainty, via variations on methods such as cross-validation, such as leave-one-out, leave-future-out, leave-group-out, 5-fold, 10-fold, and quantified via metrics such as expected log posterior predictive density, Watanabe Akaike information criteria, root mean squared error, R2, adjusted R2, Brier scores, calibration assessed with stable reliability diagrams and CORP, net reclassification index, etc simultaneously through a food composition database and generating both absolute and relative health-effect predictions and food scorings.

11. The method of claim 10, wherein said back propagating is performed through posterior predictive distributions from selected model(s), thus including an assessment of both within and between model uncertainty, or from summary statistics of the model parameters.

12. The method of claim 9, wherein at least one predictive health model is used to quantitatively compare and prioritize multiple competing lifestyle interventions in terms of their capacity to affect a future health state.

13. The method of claim 9, wherein multiple food scoring systems are aggregated either from expert opinions represented as nutritional profiling systems from a published scientific article, from a data-driven modeling approach of claim 2, and/or a hybrid expert-data driven food scoring system, to generate the food recommendation data object comprised of an expert consensus evaluation, which can be defined by created a summary distribution of health scores for any given food.

14. The method of claim 9, wherein a respective uncertainty is transmitted with each of the food recommendation data object—expressed as a statistical distribution and summary statistics derived thereof representing variations in food quality or healthfulness across multiple food health or nutrition profiling systems, the dietary recommendation data object—expressed as a statistical distribution and summary statistics derived thereof representing variations in diet quality across multiple diet quality indices/patterns/metrics—, and the predicted effect data object—expressed as a statistical distribution and summary statistics derived thereof representing variations in expected effects.

15. The method of claim 9, wherein to generate the food recommendation data object, the processor uses data-driven scoring systems, and/or expert-consensus based scoring systems, which are defined by aggregating individual expert opinions about what constitutes a healthy food codified into a nutritional profiling algorithm, by combining them into a meta-nutritional profiling system.

16. The method of any of claim 9, wherein to generate the food recommendation data object, the dietary recommendation data object, and the predicted effect data object, the processor utilizes user-specific relative beliefs.
